(19) United States
(12) Patent Application Publication
(10) Pub. No.: US 2022/0290111 A1
(43) Pub. Date: Sep. 15, 2022

(54) NUCLEIC ACIDS, PROTEINS AND PROCESSES FOR PRODUCING AMIDES

(71) Applicant: Leibniz-Institut für Pflanzenbiochemie, Halle (Saale) (DE)

(72) Inventors: Thomas Vogt, Gerbstedt (DE); Arianne Schnabel, Halle (DE); Benedikt Athmer, Halle (DE); Kerstin Manke, Halle (DE)

(21) Appl. No.: 17/632,819

(22) PCT Filed: Sep. 4, 2020
(86) PCT No.: PCT/EP2020/060165

§ 371 Date: Feb. 4, 2022

(30) Foreign Application Priority Data
Sep. 5, 2019 (EP) 19195598.8

(51) Int. Cl. C12N 9/10; C12N 15/10

(52) U.S. Cl. CPC C12N 9/1029; C12Y 203/01145; C12N 15/102; C12N 2320/50

(57) ABSTRACT

The invention provides polynucleotides and nucleic acid molecules encoding piperine synthases and proteins and enzyme functional for converting piperoyl-CoA and piperidine to piperine. The invention also provides a method of producing an amide from an acyl-CoA and an amine, and to a method of producing an amide from a carboxylic acid and an amine.

### FIELD OF THE INVENTION

The present invention relates to polynucleotides and nucleic acid molecules encoding piperine synthases and to proteins and enzyme functional for converting piperoyl-CoA and piperidine to piperine and/or cis/trans isomers thereof. The invention also relates to a method of producing an amide from an acyl-CoA and an amine, and to a method of producing an amide from a carboxylic acid and an amine.

### BACKGROUND OF THE INVENTION

Piperamides constitute a class of natural compounds, where aromatic or aliphatic acids are linked to primary or secondary amines by an amide bond. Whereas numerous piperamides have been isolated from various members of the Piperaceae, the biosynthesis of these metabolites remained enigmatic. Piperine, the most prominent member of these compounds, is present in high amounts in Piper nigrum (black pepper) and largely responsible for the pungent taste of this economically relevant spice. In addition to the use as a spice, piperine and piperamides have been used as antimicrobial, insecticidal, fungicidal as well as health-promoting agents for all kinds of diseases in the ancient world. Whereas previous publications refer to the identification, the chemical characterization and the properties of piperine and related amides, neither a gene sequence nor a protein sequence has been reported so far (Leistner E and Spenser ID, 1973; Geisler J G and Gross G G, 1990). The latter authors reported a piperine synthase activity (EC 2.3.1.145) in crude protein extracts of black pepper shoots, which was able to convert piperoyl-CoA and piperidine to piperine. Due to the apparent instability of the activity, purification of a protein having the activity was not achieved and the catalytic activity of the instable enzyme preparation was rather low. As a consequence, the biotechnological production of piperine of piperamides has not been possible.

Piperamides are known to interact with the human TRPV-vanilloid receptor (McNamara F N et al., 2006), which is involved in pain perception. In addition, piperine stabilizes other voltage-gated sodium channels (Suzuki T and Yamato S, 2018) and is able to inhibit MDR-1 type ABC-transporters, preventing the export of chemotherapeutic agents from cancer cells and thereby reducing the toxicity and exposure of these compounds to healthy human cells (Li H et al., 2018). Synthetic piperlongumine, originally isolated from Piper longum is known to selectively kill tumor cells and has been proposed as selective acetylcholine esterase inhibitors to be used in the treatment of Alzheimer disease (Wiemann et al., 2017). Therefore, piperine synthase-associated enzymes, if found, could serve as a blueprint to develop and engineer a new type of plant derived natural products as potent drugs, also in combination with corresponding CoA-ligases, cytochrome P450 monooxygenases, acyl-, glycosyl-, or methyltransferases. Further, such enzymes could promote the production of natural piperamides and related compounds under controlled conditions in a safe and sustainable way, in combination with, but independent from, chemical synthesis.

Departing from the prior art, it is an object of the present invention to provide proteins, and polynucleotides encoding them, that are capable of converting piperoyl-CoA and piperidine to piperine. It is also an object of the present invention to provide proteins, preferably pure or purified proteins, capable of converting piperoyl-CoA and piperidine to piperine that are stable, have high catalytic activity, and where activity is clearly defined on the basis of amino acid sequence identity. It is also an object of the present invention to provide a method of producing an amide from an acyl-CoA and an amine, and proteins and enzymes therefor. It is also an object of the present invention to provide products that can be synthesized by the enzymes disclosed herein.

### SUMMARY OF THE INVENTION

For accomplishing these objects, the invention provides:
1) A polynucleotide the nucleotide sequence of which is:
   (a) the nucleotide sequence defined in SEQ ID NO: 1, or
   (b) a nucleotide sequence of at least 93% sequence identity to SEQ ID NO: 1, or
   (c) a nucleotide sequence encoding an amino acid sequence comprising or consisting of the amino acid sequence defined in SEQ ID NO: 2, or
   (d) a nucleotide sequence encoding an amino acid sequence of at least 70% sequence identity to the amino acid sequence defined in SEQ ID NO: 2, or
   (e) a nucleotide sequence encoding an amino acid sequence of at least 75% sequence similarity to the amino acid sequence defined in SEQ ID NO: 2, or
   (f) a nucleotide sequence encoding an amino acid sequence of from 1 to 138 amino acid substitutions, additions, insertions and/or deletions compared to the amino acid sequence defined in SEQ ID NO: 2, or
   (g) a fragment of the nucleotide sequence defined in any one of items (a) to (f), the fragment being at most 42 nucleotides shorter due to 5′- and/or 3′-terminal deletions of the nucleotide sequence defined in any one of items (a) to (f);
   or a polynucleotide as defined above but further containing one or more introns.
2) A polynucleotide the nucleotide sequence of which is:
   (a′) the nucleotide sequence defined in SEQ ID NO: 3, or
   (b′) a nucleotide sequence of at least 93% sequence identity to SEQ ID NO: 3, or
   (c′) a nucleotide sequence encoding an amino acid sequence comprising or consisting of the amino acid sequence defined in SEQ ID NO: 4, or
   (d′) a nucleotide sequence encoding an amino acid sequence of at least 70% sequence identity to the amino acid sequence defined in SEQ ID NO: 4, or
   (e′) a nucleotide sequence encoding an amino acid sequence of at least 75% sequence similarity to the amino acid sequence defined in SEQ ID NO: 4, or
   (f′) a nucleotide sequence encoding an amino acid sequence of from 1 to 138 amino acid substitutions, additions, insertions and/or deletions compared to the amino acid sequence defined in SEQ ID NO: 4, or
   (g′) a fragment of the nucleotide sequence defined in any one of items (a′) to (f′), the fragment being at most 42 nucleotides shorter due to 5′- and/or 3′-terminal deletions of the nucleotide sequence defined in any one of items (a′) to (f′); or a polynucleotide as defined above but further containing one or more introns.
3) Nucleic acid molecule, recombinant construct, or vector comprising the polynucleotide defined in 1) or 2).
4) The nucleic acid molecule, recombinant construct, or vector according to 3), further comprising a polynucleotide, nucleic acid or gene encoding a CoA-ligase.
5) A prokaryotic or eukaryotic cell comprising the nucleic acid molecule or vector according to 3) or 4), and optionally a further polynucleotide encoding a CoA-ligase.
6) A protein comprising a polypeptide the amino acid sequence of which
   (i) is or comprises the amino acid sequence defined in SEQ ID NO: 2, or
   (ii) comprises or consists of an amino acid sequence that has at least 70% sequence identity to the amino acid sequence defined in SEQ ID NO: 2, or
   (iii) comprises or consists of an amino acid sequence that has at least 75% sequence similarity to the amino acid sequence defined in SEQ ID NO: 2, or
   (iv) comprises or consists of an amino acid sequence of from 1 to 138 amino acid substitutions, additions, insertions and/or deletions compared to the amino acid sequence defined in SEQ ID NO: 2, or
   (v) is a fragment of the amino acid sequence defined in any one of items (i) to (iv), the fragment being at most 14 amino acid residues shorter due to N- and/or C-terminal deletions of the amino acid sequence defined in any one of items (i) to (iv).
7) A protein comprising a polypeptide the amino acid sequence of which
   (i′) is or comprises the amino acid sequence defined in SEQ ID NO: 4, or
   (ii′) comprises or consists of an amino acid sequence that has at least 70% sequence identity to the amino acid sequence defined in SEQ ID NO: 4, or
   (iii′) comprises or consists of an amino acid sequence that has at least 75% sequence similarity to the amino acid sequence defined in SEQ ID NO: 4, or
   (iv′) comprises or consists of an amino acid sequence of from 1 to 138 amino acid substitutions, additions, insertions and/or deletions compared to the amino acid sequence defined in SEQ ID NO: 4.
8) The protein according to 6), wherein the sequence identity is at least 75%, preferably at least 80%, more preferably at least 85%, even more preferably at least 90%, even more preferably at least 95%, and most preferably at least 97%; and/or
   the sequence similarity is at least 85%, preferably at least 85%, more preferably at least 90%, even more preferably at least 95%, and most preferably at least 97%; and/or
   wherein the number of said amino acid substitutions, additions, insertions and/or deletions is from 1 to 100, preferably from 1 to 70, more preferably from 1 to 40, more preferably from 1 to 20, and most preferably from 1 to 10 compared to the amino acid sequence defined in SEQ ID NO: 2.
9) The protein according to 7), wherein the sequence identity is at least 75%, preferably at least 80%, more preferably at least 85%, even more preferably at least 90%, even more preferably at least 95%, and most preferably at least 97%; and/or
   the sequence similarity is at least 85%, preferably at least 85%, more preferably at least 90%, even more preferably at least 95%, and most preferably at least 97%; and/or
   wherein the number of said amino acid substitutions, additions, insertions and/or deletions is from 1 to 100, preferably from 1 to 70, more preferably from 1 to 40, more preferably from 1 to 20, and most preferably from 1 to 10 compared to the amino acid sequence defined in SEQ ID NO: 4.
10) The protein according to any one of 6) or 8) or the protein encoded by the nucleic acid molecule of 1), comprising the following amino acid sequence stretch in the one-letter code: DWGWG; and/or
   wherein the amino acid residue at the position corresponding to position 107 in SEQ ID NO: 2 is C.
11) The protein according to 7) or 9) or the protein encoded by the nucleic acid molecule of 2), comprising the following amino acid sequence stretch in the one-letter code: DWGWG; and/or
   wherein the amino acid residue at the position corresponding to position 106 in SEQ ID NO: 4 is C.
12) The protein according to any one of 6), 8) and 10) or the protein encoded by the polynucleotide of 1), that is an enzyme functional for converting piperoyl-CoA and piperidine to piperine, or to isopiperine and/or chavicine.
13) The protein according to 12), wherein said protein is an enzyme having a catalytic activity of converting piperoyl-CoA and piperidine to isopiperine and/or chavicine preferably with a total catalytic activity (for both products) of at least 0.56, preferably at least 1.6, more preferably at least 2.4, even more preferably at least 3.2, and most preferably of at least 9.6 nkat/mg total protein at pH=8.0 at 30° C. in 30 mM Tris-HCl with initial substrate concentrations of 500 μM piperoyl-CoA and 20 mM piperidine, and 1 mM dithiothreitol (DTT). More preferably, it produces isopiperine with a catalytic activity of at least 0.56, preferably at least 1.6, more preferably at least 2.4, even more preferably at least 3.2, and most preferably of at least 9.6 nkat/mg total protein under the conditions given in the previous sentence.
14) The protein according to 12), wherein said protein is an enzyme having a catalytic activity of converting piperoyl-CoA and piperidine to piperine with a catalytic activity of at least 0.14, preferably at least 0.4, more preferably at least 0.6, even more preferably at least 0.8, even more preferably at least 2.4 nkat/mg total protein at pH 8.0 at 30° C. in 30 mM Tris-HCl buffer containing 1 mM dithiothreitol (DTT). Initial substrate concentrations are preferably 500 μM piperoyl-CoA and 20 mM piperidine.
15) The protein according to any one of the 7), 9) or 11) or the protein encoded by the polynucleotide of 2), that is an enzyme functional for converting piperoyl-CoA and piperidine to piperine.
16) The protein according to 7), 9), 11) or 15), wherein said protein is an enzyme having a catalytic activity for converting piperoyl-CoA and piperidine to piperine with a catalytic activity of at least 0.14, preferably at least 0.4, more preferably at least 0.8, even more preferably at least 2.4, and most preferably at least 5.0 nkat/mg total protein at pH 8.0 at 30° C. in 30 mM Tris-HCl buffer containing 1 mM dithiothreitol (DTT). Initial substrate concentrations are preferably 500 μM piperoyl-CoA and 20 mM piperidine.
17) Nucleic acid molecule encoding the protein of any one of 6) to 16), or encoding the protein of any one of 38), 39), 40), or 41).
18) A method of producing the protein according to any one of 7) to 16), comprising expressing a polynucleotide according to 1) or 2) or a nucleic acid molecule encoding a protein according to any one of 7) to 16) in a prokaryotic or eukaryotic host or host cell.
19) A process of producing an amide from an acyl-CoA and an amine that is a primary or secondary amine, comprising incubating the acyl-CoA and the amine in the presence of a protein according to any one of 6) to 16), optionally followed by isolating the amide produced.
20) The process according to 19), wherein the acyl-CoA is a C₁-C₂₀ acyl-CoA, preferably a C₃-C₁₈ acyl-CoA, more preferably a C₄-C₁₆ acyl-CoA, and most preferably a C₅-C₁₄ acyl-CoA.
21) The process according to any one of 19) to 20), wherein the amine is a C₁-C₁₄ amine, preferably a C₂-C₁₂ amine, preferably a C₃-C₁₀ amine, and most preferably a C₅-C₈ amine; and/or the amine is a hydrocarbylamine, a heterohydrocarbylamine, a di-N-hydrocarbylamine, a N-hydrocarbyl-N-heterohydrocarbyl-amine, or a di-N-heterohydrocarbylamine. 22) The process according to 21), wherein said heterohydrocarbyl group has from 1 to 3 heteroatoms selected from O, N or S, preferably O; or has 1 or 2 heteroatoms selected from O, N or S, preferably O.
23) The process according to any one of 19), 20), 21) to 22), wherein the amine is a linear or branched primary amine or it is a cyclic secondary amine, preferably an alicyclic secondary amine.
24) The process according to any one of 19) to 23), wherein the amine is a substituted or unsubstituted:
   alkylamine, di-N-alkylamine, cycloalkyl-amine, heterocycloalkyl-amine, N-alkyl-N-cycloalkyl-amine, cycloalkenylamine, N-alkyl-N-cycloalkenyl-amine, N-cycloalkylalkylamine, N-alkyl-N-cycloalkylalkylamine, aniline, N-alkyl-aniline, N-phenylalkylamine, N-alkyl-N-phenylalkyl-amine, wherein these substituted or unsubstituted compounds preferably have from 2 to 14 carbon atoms.
25) The process according to any one of 19) to 23), wherein the amine is a substituted or unsubstituted four-, five-, six-, or seven-membered nitrogen-containing ring such as azetidine, pyrrole, pyrroline, pyrrolidine, imidazole, imidazoline, oxazole, oxazoline, oxazolidine, pyridine, dihydropyridine, piperidine, piperazine, or morpholine, wherein these substituted or unsubstituted compounds preferably have from 2 to 14 carbon atoms.
26) The process according to any one of 19) to 25), wherein the acyl-CoA is a compound of the following general formula (1):

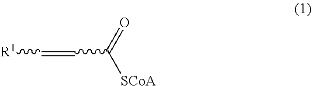

preferably of the following general formula (3):

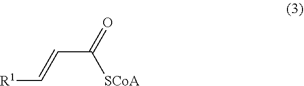

wherein R¹ is a C₁-C₁₇ hydrocarbyl group or C₂-C₁₆ heterohydrocarbyl group, and SCoA is a CoA group.
27) The process according to any one of 19) to 26), wherein the acyl-CoA is a compound of the following general formula (5):

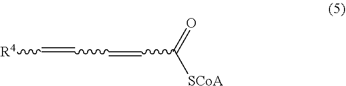

preferably the acyl-CoA is a compound of the following general formula (7):

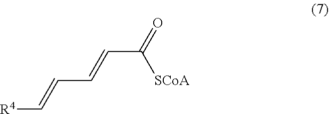

wherein R⁴ is a C₁-C₁₅ hydrocarbyl group or C₂-C₁₄ heterohydrocarbyl group, and SCoA is a CoA group.
28) The process according to any one of 19) to 27), wherein the acyl-CoA is a compound of the following general formula (9):

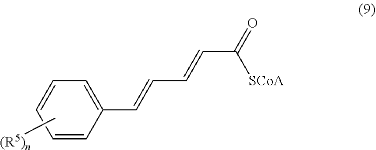

wherein n is an integer of from 1 to 4, preferably 1 or 2, and multiple groups R⁵ may be the same or different, R⁵ is selected from C₁ to C₆ alkyl, C₂ to C₆ alkenyl, C₂ to C₆ alkynyl, C₁ to C₆ alkoxy, hydroxy, or hydroxy C₁ to C₆ alkyl, and sulfhydryl, or two R₅ at adjacent carbon atoms of the phenyl ring together form a five- or six-membered ring, such as a 3,4-methylene dioxy group, and SCoA is a CoA group.
29) The process according to any one of 19), 20), 21), 22), 23), 24) or 25), wherein the acyl-CoA is an alkanoyl-CoA, preferably a C₁ to C₁₄ alkanoyl-CoA.
30) The process according to any one of 19) to 28), wherein the acyl-CoA is piperoyl-CoA, (E)-8-methyl-6-nonenoyl-CoA, 5-phenylpentanoyl-CoA, or 3,4-methylenedioxy cinnamoyl-CoA, and the primary or secondary amine is selected from the group consisting of piperidine, pyrrolidine, isobutylamine, benzylamine, 4-aminomethylpiperidine, vanillylamine, and 1,7-diamino-n-heptane, preferably the acyl-CoA is (E)-8-methyl-6-nonenoyl-CoA, the amine is vanillylamine, and the amide is capsaicin.
31) The process according to any one of 19) to 30), wherein the protein is a protein as defined in any one of 7), 9) and 11) or the protein encoded by the polynucleotide of 2).
32) The process according to any one of 19) to 31), wherein piperine or chavicine is produced from piperoyl-CoA and piperidine.
33) The process according to any one of 19) to 32), which is carried out in the presence of prokaryotic or eukaryotic cells expressing the protein according to any one of 6) to 16) or in a prokaryotic or eukaryotic cell according to 5).
34) The process according to any one of 19) to 33), wherein the reaction of piperoyl-CoA with piperidine to piperine is excluded.
35) A process of producing an amide from a carboxylic acid and an amine that is a primary or secondary amine, comprising incubating the carboxylic acid and the amine in the presence of a CoA ligase, coenzyme A, and a protein according to any one of 6) to 16), optionally followed by isolating the amide produced.
36) The process according to 35), wherein the amine is as defined in any one of 21) to 25) and the carboxylic acid is a compound having the CoA moiety of any of the CoA-esters defined in 20) or 26) to 30) replaced by a hydroxyl moiety.
37) The process according to 35) or 36), wherein said process is carried out in, or in the presence of, a prokaryotic or eukaryotic cell expressing said CoA ligase and said protein.
38) A protein, or protein composition comprising said protein, said protein or protein composition having a catalytic activity for converting piperoyl-CoA and piperidine to piperine with a catalytic activity of at least 0.8, preferably at least 2.4, and most preferably at least 5.0 nkat/mg of said protein or protein composition, respectively, at 30° C. in 30 mM Tris-HCl buffer pH 8.0, containing 1 mM dithiothreitol (DTT), with substrate concentrations of 500 μM piperoyl-CoA and 20 mM piperidine.
39) The protein according to 38), 6), 7), 8) or 9), or a protein encoded by the polynucleotide of 1) or 2), comprising a polypeptide comprising the following amino acid residues or amino acid sequence stretches in the one-letter code:
   L at the position corresponding to amino acid residue 42 in SED ID NO: 2 or at the position corresponding to amino acid residue 43 in SED ID NO: 4,
   DVG at the position corresponding to amino acid residues 396 to 398 in SED ID NO: 2 or at the position corresponding to amino acid residues 400 to 402 in SED ID NO: 4,
   FLAT at the position corresponding to amino acid residues 404 to 407 in SED ID NO: 2 or at the position corresponding to amino acid residues 408 to 411 in SED ID NO: 4,
   ML at the position corresponding to amino acid residues 312 to 313 in SED ID NO: 2 or at the position corresponding to amino acid residues 316 to 317 in SED ID NO: 4,
   N at the position corresponding to amino acid residue 366 in SED ID NO: 2 or at the position corresponding to amino acid residue 370 in SED ID NO: 4,
   HXXXD at the position corresponding to amino acid residues 168 to 172 in SED ID NO: 2 or at the position corresponding to amino acid residues 168 to 172 in SED ID NO: 4,
   DWGWG at the position corresponding to amino acid residues 383 to 387 in SED ID NO: 2 or at the position corresponding to amino acid residues 387 to 391 in SED ID NO: 4,
   F at the position corresponding to amino acid residue 130 in SEQ ID NO: 2 or at the position corresponding to amino acid residue 129 in SEQ ID NO: 4, and/or
   K at the position corresponding to amino acid residue 133 in SEQ ID NO: 2 or at the position corresponding to amino acid residue 132 in SEQ ID NO: 4;
   and optionally as further defined in any one of 8) to 16).
40) The protein according to 38), 39), 6), or 8), or a protein encoded by the polynucleotide of 1), wherein the protein comprises a polypeptide comprising one or more or all of the following amino acid residues or amino acid sequence stretches in the one-letter code:

LFLTAI at the position corresponding to amino acid residues 42 to 47 in SEQ ID NO: 2,

GLML at the position corresponding to amino acid residues 310 to 313 in SEQ ID NO: 2,

SN at the position corresponding to amino acid residues 365 to 366 in SEQ ID NO: 2, and/or

LID at the position corresponding to amino acid residues 376 to 378 in SEQ ID NO: 2.
41) The protein according to 38), 39), 7), or 9) or a protein encoded by the polynucleotide of 2), wherein the protein comprises a polypeptide comprising one or more or all of the following amino acid residues or amino acid sequence stretches in the one-letter code:
   LHISGF at the position corresponding to amino acid residues 43 to 48 in SEQ ID NO: 4,
   SIML at the position corresponding to amino acid residues 314 to 317 in SEQ ID NO: 4,
   TN at the position corresponding to amino acid residues 369 to 370 in SEQ ID NO: 4, and/or
   LVE at the position corresponding to amino acid residues 380 to 382 in SEQ ID NO: 4.
42) The polynucleotide, nucleic acid molecule, recombinant construct, or vector according to 1), 2), 3), or 4), encoding a protein as defined in 38), 39), 40) or 41), preferably according to 38).
43) The polynucleotide according to 2) or a nucleic acid molecule, recombinant construct, or vector comprising a polynucleotide according to 2), encoding a protein as defined in 38), 39), or 41).

The inventors have found enzymes from immature black pepper fruits that catalyze the decisive step in piperine formation from piperoyl CoA and piperidine. Identification of piperine synthase was achieved based on three assumptions conceived by the inventors. First, the biosynthetic enzymes involved in piperine and piperamide formation are highly expressed in maturing fruits. Second, piperine synthase is expressed differentially in fruits, leaves, and flowers, with highest expression anticipated for young fruits, and third are specifically detected in black pepper and related Piperaceae and rely on activated CoA esters.

Based on in-house analysis, data assembly, and interpretation of transcriptome data according to above assumptions from immature fruits of black pepper, and relative transcript abundance, the inventors identified individual polynucleotides and genes involved in the synthesis of piperine and functionally expressed the corresponding cDNAs in Escherichia coli (E. coli) cells. The resulting heterologous enzymes encoded by these genes were capable of synthesizing the natural product piperine from piperoyl-coenzyme A (piperoyl-CoA) and piperidine. The inventors have surprisingly found the affinity of the recombinant enzymes for the amine substrate is somewhat low, whereby initial attempts to detect enzymatic activity had failed. Once this was established, the activity of the enzymes allowed efficient production of piperine. In addition, the promiscuity of the isolated enzymes allowed biosynthesis of various amides such as piperamides that would, otherwise, require organic synthesis (Bauer A et al. 2019; Fregnan A M et al., 2017).

The production of piperine in biotechnological processes through recombinant enzymes can be considered “natural production” and any product derived from such processes are considered as “GRAS” (generally recognized as safe) by the Food and Drug Association (FDA) in the USA. This usually facilitates the approval as a dietary component or its use as a drug, or health, specifically skin care preparations. The proteins provided herein can be used for synthesizing a wide range of amides such as piperamides by, for example, microbial fermentation. Such compounds have potential relevance as precursors for active agents of medicaments and in the treatment of diseases as demonstrated for vitiligo and cancer (Mih\check{a}il\check{a} B et al. 2019, Xie Z et al., 2019). In addition, such compounds potentially represent alternative insecticides (Navickiene D H M et al. 2007). Targeted mutagenesis allows further expansion of the substrate spectrum of the enzymes described herein. Combinatorial expression with further, known enzymes allows the use of substrate precursors in the synthesis process and a further refinement of the end product.

### BRIEF DESCRIPTION OF THE DRAWINGS

FIG. 1: Dendrogram of various BAHD-like enzyme sequences. The piperine synthases PipSynthase1 and Pipsynthase 2, marked in bold cluster within, but are clearly distinct from, benzoyl-benzoate transferases that are characterized in the case of Clarkia breweri. Crystallized BAHD-like enzymes depicted in bold and marked by the pdb accession number (www.rcsb.org/) are only distantly related to PipSynthase1 and 2 with respect to the protein sequence and the enzymatic function.

FIG. 2: Amino acid sequence comparison of four sequences, PipSynthase 1 (SEQ ID NO: 2), PipSynthase 2 (SEQ ID NO: 4), their closest functionally annotated homologue, Benzoyl-Benzoate Transferase (BBT) vom Clarkia breweri (SEQ ID NO: 12) (see FIG. 1), and the first crystallized BAHD, Vinorine Synthase (VS, SEQ ID NO: 13), illustrates the high (61.5%) sequence identity between PipSynthase 1 and 2, compared to 42% to BBT and only 18% to VS. The HXXXD-motif (SEQ ID NO:5), and the DWGWG-motif (SEQ ID No: 6) are marked.

FIG. 3: Purification of recombinant PipSynthase 1 (A) and PipSynthase 2 (B) by affinity chromatography on Ni-NTA resin (Macherey-Nagel) documented by SDS-PAGE. A. 1, crude E. coli (LEMO cells) protein extract containing recombinant PipSynhase 1; 2, Ni NTA unbound fraction; 3, 30 mM imidazole wash fraction; 4, 50 mM imidazole wash fraction; 5-7 5 μl out of 1.5 ml each, 300 mM imidazole fractions eluting purified PipSynthase 1. B. 1, crude E. coli (LEMO cells) protein extract containing recombinant PipSynthase 2; 2, 30 mM imidazole wash fraction; 3, 50 mM imidazole wash fraction; 4-6, 5 μl out of 1.5 ml each, 300 mM imidazole fractions eluting purified PipSyn 2. M, molecular weight marker, bands of 72 kDa, 55 kDa, and 37 kDa are marked.

FIG. 4 HPLC-chromatograms illustrating formation of piperine and configurational isomers, isopiperine and chavicine by PipSynthase 1 (A) and piperine by PipSynthase 2 (B), in both cases from piperoyl CoA and piperidine by a purified, recombinant enzyme preparation described in FIG. 2. 50 μl assays (as described) were stopped after 2 min (PipSynthase 1) and 5 min (PipSynthase 2) with a mix of 10 μl 1% formic acid/acetonitrile (1:1), centrifuged and 10 μl of the supernatant separated on a 5 cm Nucleoshell RP18 column (Macherey and Nagel) at a flow rate of 0.6 ml/min with a gradient of increasing acetonitrile concentrations (marked in grey) (solvent B) in water/0.1% formic acid (solvent A). Piperine and its isomers isopiperine and chavicine were identified by LC-MS detection at m/z 286.1 (QDA mass detector, Waters) in a positive ionization mode based on a piperine standard (Sigma) and in parallel by UV-detection and absorbance maxima of 340 nm (piperine), 330 nm (isopiperine), and 320 nm (chavicin) by a photodiode array detector (Waters) in the range of 320-370 nm.

FIG. 5 Example of substrate preference/promiscuity of PipSynthase 2. Piperoyl CoA (200 μM) and three different amines (piperidine, pyrrolidine, isobutylamine) at concentrations of 4 mM were used as substrates resulting in the formation of piperine (1), piperoylpyrrolidine (2), piperoylisobutylamine (3), corresponding molecular masses m/z are indicated, recorded in positive ionization mode.

FIG. 6 Example of non-enzymatic and enzymatic product formation at the indicated conditions with 1 μg PipSyn1, 500 μM piperoyl CoA, and 20 mM concentration of piperidine or benzylamine at pH 8.5. The assay was run up to 30 min to demonstrate spontaneous product formation, 8% in case of piperidine, 3.5% in case of benzylamine of final product formation. Detection by LC-MS in pos. mode as described.

FIG. 7 Sequence analysis of PipSynthase 1 and 2 (SEQ ID NOs: 2 and 4, respectively) in comparison to related BAHD-like enzymes. Enzymatic function of the vinorine synthase (acetyltransferase involved in indole alkaloid formation) (SEQ ID NOs: 13) and the benzoyl-benzoate transferase (required for volatile formation) from Clarkia (SEQ ID NOs: 12) are experimentally confirmed. Enzymatic function of PUN1 (SEQ ID NOs: 20), a capsaicin synthase from Capsicum spec. and of a benzoyl-benzoate transferase from Ziziphus (SEQ ID NOs: 19) are predicted based on the presence of the conserved HxxxD and DFGWG motifs. Specific regions and amino acid residues (given in bold letters) of envisaged relevance for piperine synthase activity and piperine formation are marked in grey. Also shown is the amino acid sequence of the shikimate O-hydroxycinnamoyltransferase from Arabidopsis thaliana (SEQ ID NOs: 21).

### DETAILED DESCRIPTION OF THE INVENTION

The inventors of the present invention have identified the coding sequence and the amino acid sequence of a protein termed PipSynthase2 that has enzymatic activity in converting piperoyl-CoA and piperidine to piperine. The inventors have also identified the coding sequence and the amino acid sequence of a protein termed PipSynthase1 that has enzymatic activity in converting piperoyl-CoA and piperidine to piperine, but also to its configurational isomers isopiperine and chavicine. The coding sequence and amino acid sequence of PipSynthase2 are those given in SEQ ID NOs: 3 and 4, respectively, disclosed herein. The coding sequence and amino acid sequence of PipSynthase1 are those given in SEQ ID NOs: 1 and 2, respectively. The terms “PipSynthase1” and “PipSynthase2” are also used herein for proteins comprising the amino acid sequence of SEQ ID NOs: 2 and 4, respectively, and optionally additional sequence stretches like purification tags (e.g. a His-tag).

### Polynucleotides and Nucleic Acids of the Invention

The invention provides a polynucleotides of a first and of a second general embodiment. Those of the first general embodiment are described in the following with reference to SEQ ID NO: 1 and 2. Those of the second general embodiment as described in the following with reference to SEQ ID NO: 3 and 4.

Accordingly, the invention provides a polynucleotide the nucleotide sequence of which is:

(a) the nucleotide sequence defined in SEQ ID NO: 1, or

(b) a nucleotide sequence of at least 93% sequence identity to SEQ ID NO: 1, or

(c) a nucleotide sequence encoding an amino acid sequence comprising or consisting of the amino acid sequence defined in SEQ ID NO: 2, or

(d) a nucleotide sequence encoding an amino acid sequence of at least 70% sequence identity to the amino acid sequence defined in SEQ ID NO: 2, or

(e) a nucleotide sequence encoding an amino acid sequence of at least 75% sequence similarity to the amino acid sequence defined in SEQ ID NO: 2, or

(f) a nucleotide sequence encoding an amino acid sequence of from 1 to 138 amino acid substitutions, additions, insertions and/or deletions compared to the amino acid sequence defined in SEQ ID NO: 2, or

(g) a fragment of the nucleotide sequence defined in any one of items (a) to (f), the fragment being at most 42, preferably at most 21, nucleotides shorter due to 5′- and/or 3′-terminal deletions of the nucleotide sequence defined in any one of items (a) to (f).

Also disclosed is a polynucleotide as defined above but further containing one or more introns.

Further, the invention provides a polynucleotide the nucleotide sequence of which is:

(a′) the nucleotide sequence defined in SEQ ID NO: 3, or

(b′) a nucleotide sequence of at least 93% sequence identity to SEQ ID NO: 3, or

(c′) a nucleotide sequence encoding an amino acid sequence comprising or consisting of the amino acid sequence defined in SEQ ID NO: 4, or

(d′) a nucleotide sequence encoding an amino acid sequence of at least 70% sequence identity to the amino acid sequence defined in SEQ ID NO: 4, or

(e′) a nucleotide sequence encoding an amino acid sequence of at least 75% sequence similarity to the amino acid sequence defined in SEQ ID NO: 4, or

(f′) a nucleotide sequence encoding an amino acid sequence of from 1 to 138 amino acid substitutions, additions, insertions and/or deletions compared to the amino acid sequence defined in SEQ ID NO: 4, or

(g′) a fragment of the nucleotide sequence defined in any one of items (a′) to (f′), the fragment being at most 42, preferably at most 21, nucleotides shorter due to 5′- and/or 3′-terminal deletions of the nucleotide sequence defined in any one of items (a′) to (f′). Also disclosed is a polynucleotide as defined above but further containing one or more introns.

The polynucleotide of the invention is a polymer of nucleotides linked by phosphodiester groups. The polynucleotide may be DNA or RNA or may comprise both ribo- and deoxyribonucleotides. The polynucleotide may further comprise nucleotide moieties having modifications at the pentose moiety, the base and/or the phosphodiester moieties. Preferably, the polynucleotide is DNA or RNA.

Herein, the determination of (nucleotide sequence) and amino acid sequence identities is done using the DNASTAR Lasergene software package, using Clustal V as the default algorithm of the MegAlign tool. Herein, where the protein or the amino acid sequence is defined by a number or numerical range of amino acid substitutions, additions, insertions and/or deletions, these amino acid substitutions, additions, insertions or deletions may be combined, but the given number or numerical range refers to the sum of all amino acid substitutions, additions, insertions and deletions. Among amino acid substitutions, additions, insertions and deletions, amino acid substitutions, additions, and deletions are preferred. The term “insertion” relates to insertions within the amino acid sequence of the reference sequence, i.e. excluding additions at the C- or N-terminal end. The term “addition” means additions at the C- or N-terminal end of the amino acid sequence of the reference sequence. A deletion may be a deletion of a terminal or an internal amino acid residue of a reference sequence. A reference sequence is the sequence of the given SEQ ID NO.

The wording “nucleotide sequence of at least x % sequence identity to SEQ ID NO: y means that the nucleotide sequence has at least the same number of nucleotides as the reference sequence of SEQ ID NO: y and has the indicated minimum sequence identity x over the entire length of SEQ ID NO: y. y is an integer that generically refers to the sequences of the SEQ ID NOs of the invention.

The wording “amino acid sequence of at least x % sequence identity to the amino acid sequence defined in SEQ ID NO: y means that the amino acid sequence has at least the same number of amino acid residues as the reference sequence of SEQ ID NO: y and has the indicated minimum sequence identity x over the entire length of SEQ ID NO: y. The wording “amino acid sequence of at least x % sequence similarity to the amino acid sequence defined in SEQ ID NO: y means that the amino acid sequence has at least the same number of amino acid residues as the reference sequence of SEQ ID NO: y and has the indicated minimum sequence similarity x over the entire length of SEQ ID NO: y.

The invention also provides specific polynucleotides and nucleic acids encoding the proteins and embodiments described in the following section.

The invention further provides a nucleic acid molecule comprising a polynucleotide of the invention. The nucleic acid molecule may be a recombinant construct, a plasmid, or a vector, that are further described below.

The polynucleotides of the invention may encode a protein of the invention, as described in more detail in the following, and may have an enzymatic activity as also described in the following. Generally, a polynucleotide of the first general embodiment encodes a protein of the first general embodiment. Generally, a polynucleotide of the second general embodiment encodes a protein of the second general embodiment.

### Proteins of the Invention

The invention provides two general classes of proteins, referred to herein as the proteins of the first and second general embodiment. The proteins of the first general embodiment are those defined herein with reference to SEQ ID NO:2. The proteins of the second general embodiment are those defined herein with reference to SEQ ID NO:4.

In the first general embodiment, the invention provides a protein comprising a polypeptide, the amino acid sequence of said polypeptide

(i) is or comprises the amino acid sequence defined in SEQ ID NO: 2, or

(ii) comprises or consists of an amino acid sequence that has at least 70% sequence identity to the amino acid sequence defined in SEQ ID NO: 2, or

(iii) comprises or consists of an amino acid sequence that has at least 75% sequence identity to the amino acid sequence defined in SEQ ID NO: 2, or

(iv) comprises or consists of an amino acid sequence of from 1 to 138 amino acid substitutions, additions, insertions and/or deletions compared to the amino acid sequence defined in SEQ ID NO: 2, or

(v) is a fragment of the amino acid sequence defined in any one of items (i) to (iv), the fragment being at most 14 amino acid residues shorter due to N- and/or C-terminal deletions of the amino acid sequence defined in any one of items (i) to (iv).

Preferably, a protein of the first general embodiment according to items (ii) to (v) has a catalytic activity in converting piperoyl-CoA and piperidine to piperine with a catalytic activity of at least 0.14, preferably at least 0.4, more preferably at least 0.6, even more preferably at least 0.8, and most preferably of at least 2.4 nkat/mg protein at pH=8.0 at 30° C. in 30 mM Tris-HCl, 1 mM dithiothreitol (DTT) with substrate concentrations of 500 μM piperoyl-CoA and 20 mM piperidine.

Throughout this invention, 30 mM Tris-HCl means that the concentration of Tris is 30 mM and the pH is suitably adjusted as indicated with HCl.

In the second general embodiment, the invention provides a protein comprising a polypeptide, the amino acid sequence of said polypeptide

(i′) is or comprises the amino acid sequence defined in SEQ ID NO: 4,

(ii′) comprises or consists of an amino acid sequence that has at least 70% sequence identity to the amino acid sequence defined in SEQ ID NO: 4,

(iii′) or comprises or consists of an amino acid sequence that has at least 75% sequence identity to the amino acid sequence defined in SEQ ID NO: 4, or

(iv′) comprises or consists of an amino acid sequence of from 1 to 138 amino acid substitutions, additions, insertions and/or deletions compared to the amino acid sequence defined in SEQ ID NO: 4, or

(v′) is a fragment of the amino acid sequence defined in any one of items (i′) to (iv′), the fragment being at most 14 amino acid residues shorter due to N- and/or C-terminal deletions of the amino acid sequence defined in any one of items (i′) to (iv′).

Preferably, a protein of the second general embodiment according to items (ii′) to (v′) has a catalytic activity in converting piperoyl-CoA and piperidine to piperine with a catalytic activity of at least 0.14, preferably at least 0.4, more preferably at least 0.6, even more preferably at least 0.8, and most preferably of at least 2.4 nkat/mg protein at pH=8.0 at 30° C. in 30 mM Tris-HCl, 1 mM dithiothreitol (DTT) with substrate concentrations of 500 μM piperoyl-CoA and 20 mM piperidine.

In preferred embodiments of the proteins, the sequence identity is at least 75%, preferably at least 80%, more preferably at least 85%, even more preferably at least 90%, even more preferably at least 95%, and most preferably at least 97%; and/or the sequence similarity is at least 85%, preferably at least 85%, more preferably at least 90%, even more preferably at least 95%, and most preferably at least 97%; and/or wherein the number of said amino acid substitutions, additions, insertions and/or deletions is from 1 to 100, preferably from 1 to 70, more preferably from 1 to 40, more preferably from 1 to 20, and most preferably from 1 to 10 compared to the amino acid sequence defined in SEQ ID NO: 2 or 4.

The definition given above in the section on the polynucleotides and nucleic acids regarding the determination of amino acid sequence identities and similarities, and regarding the meaning and preferred embodiments of the substitutions, additions, insertions and/or deletions also apply to the proteins of the invention.

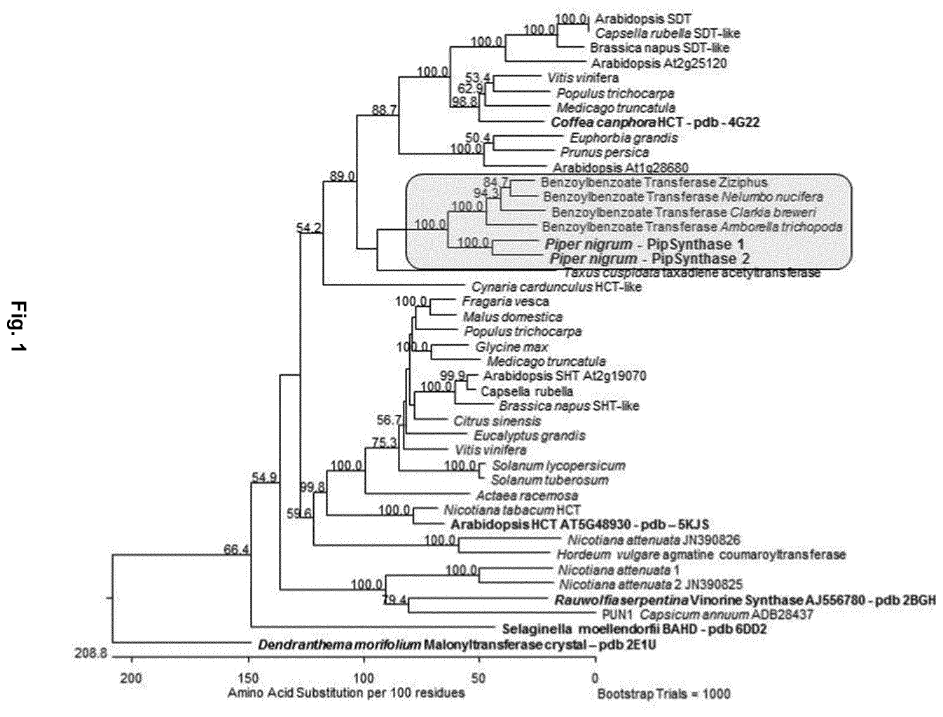

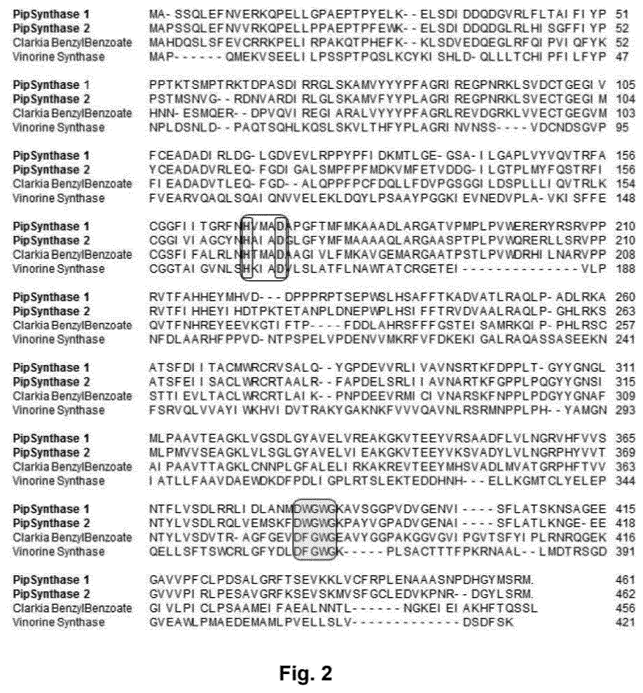

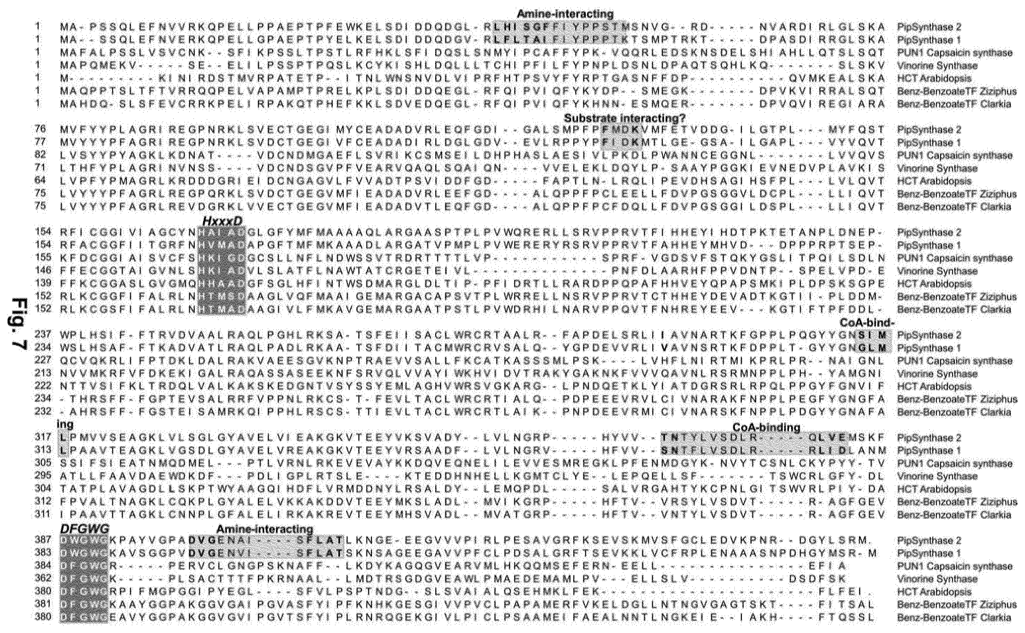

The proteins according to the invention may be classified as BAHD-like transferases (FIG. 1) and may comprise an HXXXD-motif (SEQ ID NO: 5). As for all BAHD-like transferases, the histidine in the HXXXD-motif is believed to de-protonate the amine reactant as the initial step in the reaction mechanism. The HXXXD-motif is a short sequence stretch of five standard amino acids in the one-letter code, wherein the first amino acid is a histidine and the fifth is an aspartic acid residue that flank three arbitrary standard amino acid residues depicted by X (FIG. 2, FIG. 7).

In a preferred embodiment, the proteins of the invention also comprise the amino acid stretch DWGWG (SEQ ID NO: 6) in their amino acid sequence that is different to the stretch DFGWG (SEQ ID NO: 7) present in closely related proteins depicted in FIG. 2. In one embodiment, the amino acid residue at the position corresponding to position 107 in SEQ ID NO: 2 is C, and/or the amino acid residue at the position corresponding to position 106 in SEQ ID NO: 4 is C.

The protein of the invention may be or comprise a polypeptide comprising the sequence stretch of the HXXXD-motif at the position corresponding to the position of the amino acid residues 168 to 172 in SEQ ID NO: 2 or at the position corresponding to the position of the amino acid residues 168 to 172 in SEQ ID NO: 4. Herein, the term “corresponding to” means that the amino acid sequence of the protein of the invention is aligned to the reference sequence of the respective SEQ ID NO using methods generally used in the art, for example the ClustalW program. A position in a given polypeptide corresponding to an amino acid residue or stretch of a reference sequence is the position or stretch aligning to the reference sequence.

Alternatively or additionally, the amino acid stretch DWGWG may be present, in the polypeptide of a protein of the invention, at the position corresponding to the position of the amino acid residues 383 to 387 in SED ID NO: 2 or the position corresponding to the position of the amino acid residues 387 to 391 in SED ID NO: 4.

Alternatively or additionally, the amino acid stretch FLAT (SEQ ID NO: 18) may also be present of a protein of the invention, at the position corresponding to the position of the amino acid residues 404 to 407 in SEQ ID NO: 2 or the position corresponding to the position of the amino acid residues 408 to 411 in SEQ ID NO: 4.

The protein of the invention may comprise, preferably additionally to the embodiments mentioned in the preceding paragraphs, one or more of the following amino acid residues or amino acid sequence stretches in its polypeptide (in the single-letter amino acid code) at the position corresponding to the indicated position of the reference sequences, as follows:

Y1) L at position 42 in SED ID NO: 2 or at position 43 in SED ID NO: 4
Y2) DVG from position 396 to 398 in SED ID NO: 2 or from position 400 to 402 in SED ID NO: 4,
Y3) FLAT from position 404 to 407 in SED ID NO: 2 or from position 408 to 411 in SED ID NO: 4,
Y4) ML from position 312 to 313 in SED ID NO: 2 or from position 316 to 317 in SED ID NO: 4,
Y5) N at position 366 in SED ID NO: 2 or at position 370 in SED ID NO: 4,
Y6) MOO from position 168 to 172 in SED ID NO: 2 or from position 168 to 172 in SED ID NO: 4,
Y7) DWGWG from position 383 to 387 in SED ID NO: 2 or from position 387 to 391 in SED ID NO: 4,
Y8) F at position 130 in SEQ ID NO: 2 or at position 129 in SEQ ID NO:4, and/or
Y9) K at position 133 in SEQ ID NO: 2 or at position 132 in SEQ ID NO:4.

In a preferred embodiment, the proteins of the first and the second general embodiment comprise all the amino acid residues or amino acid sequence stretches of Y1) to Y9). The amino acid residues from Y1) to Y9) may also be identified from FIG. 7.

The amino acid residues or amino acid sequence stretches Y1) to Y9) are preferably conserved in the proteins of the invention. Thus, these conserved amino acid residues allow differentiating the proteins from the first and the second general embodiment from other BADH-like proteins. A sequence alignment as depicted in FIG. 2 or FIG. 7 may be carried out to determine if the polypeptide sequence of a protein contains said amino acid residues or amino acid sequence stretches.

The proteins of the first and the second general embodiment may be distinguished from their amino acid sequences. A protein of the first general embodiment preferably has one or more of the following amino acid sequence stretches in its polypeptide (in the single-letter amino acid code) at the position corresponding to the indicated position of SEQ ID NO:2 as the reference sequence:

X1) LFLTAI (SEQ ID NO: 14) from position 42 to 47 in SEQ ID NO: 2,
X2) GLML (SEQ ID NO: 15) from position 310 to 313 in SEQ ID NO: 2,
X3) SN from position 365 to 366 in SEQ ID NO: 2, and/or
X4) LID from position 376 to 378 in SEQ ID NO: 2.

In a preferred embodiment, the protein of the first general embodiment comprises all of X1) to X4). More preferably, one, two or all of these amino acid sequence stretches are absent in proteins of the second general embodiment. The amino acid residues from X1) to X4) can also be identified in FIG. 7.

A protein of the second general embodiment preferably has one or more of the following amino acid sequence stretches in its polypeptide (in the single-letter amino acid code) at the position corresponding to the indicated position of SEQ ID NO:4 as the reference sequence:

Z1) LHISGF (SEQ ID NO: 16) from position 43 to 48 in SEQ ID NO: 4,
Z2) SIML (SEQ ID NO: 17) from position 314 to 317 in SEQ ID NO: 4,
Z3) TN of position 369 to 370 in SEQ ID NO: 4, and/or
Z4) LVE at position 380 to 382 in SEQ ID NO: 4.

In a preferred embodiment, the protein of the second general embodiment comprises all amino acid sequence stretches of Z1) to Z4). More preferably, one, two or all of these amino acid sequence stretches are absent in proteins of the first general embodiment. The amino acid residues from Z1) to Z4) can also be identified in FIG. 7.

The proteins of the present invention may have a purification tag at its N- or C-terminus for easier purification, for example with a 6×-histidine-tag, although any other purification tag is possible. This purification tag can be removed by commercially available proteases, like Factor Xa in subsequent applications.

### Enzymatic Activity of the Proteins of the Invention

The proteins defined herein with respect to SEQ ID NO: 2 and 4 preferably have catalytic activity and are thus enzymes. The catalytic activity is formation of an amide from a coenzyme A-thioester (also referred to herein as “SCoA-ester”, “CoA-ester”, or “acyl-CoA”) and an amine such as a primary or secondary amine. The designations “SCoA” and “CoA” refer to the same coenzyme A moiety and differ merely in that “SCoA” highlights the presence of the sulfur atom derived from the terminal thiol group of coenzyme A.

The recombinant proteins of the invention have a catalytic activity in converting piperoyl-CoA and piperidine to piperine, preferably with a catalytic activity of at least 0.14, preferably at least 0.4, more preferably at least 0.6, even more preferably at least 0.8, and most preferably of at least 2.4 nkat/mg total protein at pH=8.0 at 30° C. in 30 mM Tris-HCl, 1 mM dithiothreitol (DTT), preferably with substrate concentrations of 500 μM piperoyl-CoA and 20 mM piperidine. Most preferably, the protein of the second general embodiment has a catalytic activity of at least 5.0 nkat/mg total protein under the conditions defined in the previous sentence. Example 3 contains a protocol for the determination of the catalytic activity of a protein according to the invention.

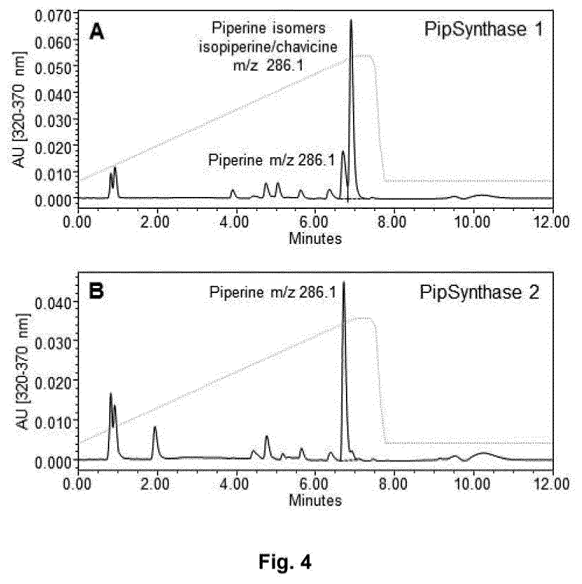

The protein of the first general embodiment may also have catalytic activity in converting piperoyl-CoA and piperidine to piperine preferably with a catalytic activity of at least 0.14, preferably at least 0.4, more preferably at least 0.6, even more preferably at least 0.8, and most preferably of at least 2.4 nkat/mg total protein at pH=8.0 at 30° C. in 30 mM Tris-HCl, 1 mM dithiothreitol (DTT), preferably with substrate concentrations of 500 μM piperoyl-CoA and 20 mM piperidine. It simultaneously and preferentially produces from the same reactants the piperine configurational isomers isopiperine and/or chavicine, preferably with a catalytic activity (with respect to both isopiperine and chavicine) of at least 0.56, preferably at least 1.6, more preferably at least 2.4, even more preferably at least 3.2, and most preferably of at least 9.6 nkat/mg total protein at pH=8.0 at 30° C. in 30 mM Tris-HCl, 1 mM dithiothreitol (DTT), preferably with substrate concentrations of 500 μM piperoyl-CoA and 20 mM piperidine. The latter catalytic activities are calculated from the sum of peak areas of isopiperine and chavicine peaks due to species of m/z 286.1 other than peaks due to piperine in a liquid chromatography (LC) trace and UV detection at 330 nm calibrated using known amounts of piperine (detected at 340 nm in LC) and assuming identical absorbance (absorption) coefficients for piperine at 340 nm, and of isopiperine and chavicine (having m/z 286.1) both at 330 nm. An assay for measuring the catalytic activity is described in the Examples and illustrated in FIG. 4.

The substrate concentrations given above and elsewhere herein are those at the beginning of a reaction or assay, i.e. initial substrate concentrations.

Standard assays may contain from at least 1 up to 10 μg of protein/50 μl, but may be raised to about 50 μg. A suitable concentration range is from 20-500 μg/ml of the protein of the invention.

As the reaction of the CoA-ester with amine can also take place non-catalyzed, the formation of product is also measured in the absence of protein, but under otherwise identical conditions, and subtracted from that measured in the presence of the protein in order to determine the enzymatic activity of a protein of the invention.

The unit kat is the SI-unit katal, wherein 1 kat is 1 mole per second. 1 nkat is one nanomole per second. The unit nkat/mg total protein is relative to the entire protein present in a sample and is thus dependent on the purity of the protein. For many applications of the proteins of the invention, it is not necessary to use highly purified preparations of the protein of the invention, provided the desired catalytic activity is obtained. The total protein in a sample can be determined using the generally known Bradford protein assay that is also described in the Examples. Usually 10-50 μl of a protein solution are mixed with 950 to 990 μl of Coomassie Brilliant Blue G-250, the Bradford Reagent (Bradford, M M, 1976) incubated for 5 minutes and measured at 595 nm against a blank solution where the protein solution is replaced by buffer. The protein of the first general embodiment may further be functional for converting piperoyl-CoA and piperidine to piperine, but preferentially to isopiperine and chavicine.

The proteins of the invention not only have catalytic activity for converting piperoyl-CoA and piperidine to piperine, but accept a wide range of other substrates to produce a wide range of different amides, as further described below. The proteins of the first and second general embodiment differ in their substrate specificity such that the protein of the first general embodiment has wider (or less specific) substrate specificity.

Further, the proteins of the invention can be used for producing amides using a wide range of conditions, as also described further below. For example, substrate concentrations tested may be from 10 μM-5 mM of the CoA ester, preferably 20 μM-500 μM of the CoA ester such as piperoyl CoA, and 100 μM to 100 mM amine, preferably 200 μM to 20 mM amine, such as piperidine. A suitable buffer other than Tris-HCl is phosphate buffer. The pH may be from 6.0 to 9.0, preferably from 7.0 to 8.5. The reaction temperature may be from 14 to 35° C., preferably from 20 to 30° C.

The proteins of the first and the second general embodiment generally differ in their catalytic activity for the turnover of CoA-esters and amines. In some reactions, a protein of the first general embodiment may show a higher catalytic activity than a protein of the second general embodiment. In other reactions, a protein of the first general embodiment may show lower catalytic activity than a protein of the second general embodiment. For example, a given protein of the invention may be assigned to the first or the second general embodiment as follows. The catalytic activity of the protein is assayed with

(i) piperoyl-CoA and piperidine as substrates and

(ii) 3,4-methylenedioxycinnamoyl-CoA and piperidine as substrates.

If the catalytic activity in terms of nkat/mg protein is higher in assay (i) than in assay (ii), the protein is generally a protein of the second general embodiment. If the catalytic activity in terms of nkat/mg protein is higher in assay (ii) than in assay (i), the protein is generally a protein of the first general embodiment. Preferably, these assays are performed in aqueous solution at pH=8.0 at 30° C. in 30 mM Tris-HCl and 1 mM dithiothreitol (DTT) with substrate concentrations of 500 μM CoA ester and 20 mM piperidine. These assays and the detection of products may be carried out as described in Example 4. Accordingly, the protein of the first general embodiment has a higher catalytic activity with 3,4-methylenedioxycinnamoyl-CoA and piperidine as substrates than with piperoyl-CoA and piperidine as substrates. The protein of the second general embodiment has a higher catalytic activity with piperoyl-CoA and piperidine as substrates than with 3,4-methylenedioxycinnamoyl-CoA and piperidine as substrates.

In these assays, the sum of all peaks in the LC trace due to species having calculated m/z values of the expected products (e.g. that of piperine in assay (i)) was used for quantification of product, as described in Example 4. In assay (i), the product(s) is quantified by comparison of the peak area of the LC-UV trace at 340 nm with the peak area of the LC-UV trace at 340 nm of (authentic) piperine. The product of assay (ii) is also quantified by comparison of the peak area of the LC-UV trace at 340 nm with that of piperine, i.e. it is assumed that the product(s) of assay (ii) and piperine have the same absorption coefficient at 340 nm. The formation of product in assay (i) and assay (ii) is preferably also measured in the absence of protein, but under otherwise identical conditions, and determined peak areas of species having calculated m/z values of the expected products are subtracted from those measured in the presence of the protein.

These embodiments of the first and second general embodiment may be combined with the embodiments described above of the respective general embodiments.

The invention also provides, in a third general embodiment, a protein, or protein composition comprising said protein, said protein or protein composition having a catalytic activity for converting piperoyl-CoA and piperidine to piperine with a catalytic activity of at least 0.8, preferably at least 2.4, and most preferably at least 5.0 nkat/mg of said protein or protein composition, respectively, at 30° C. in 30 mM Tris-HCl buffer pH 8.0, containing 1 mM dithiothreitol (DTT), with substrate concentrations of 500 μM piperoyl-CoA and 20 mM piperidine. This enzymatic activity is determined as described above, notably as described with respect to the second general embodiment and as exemplified in Example 3. The amount of non-enzymatically formed piperine is subtracted from the amount of piperine determined in the presence of protein.

The protein or protein composition of the third general embodiment may be combined with other embodiments described herein, notably with the embodiments described above for the protein of the second general embodiment. For example, the polynucleotide of the second general embodiment may encode the protein of the third general embodiment.

### Recombinant Construct and Vector

The invention further provides a recombinant construct or vector containing the polynucleotide of the invention. The recombinant construct may comprise a polynucleotide of the invention such that the latter is expressible in prokaryotic cells or eukaryotic cells or in plants to produce the protein of the invention. For this purpose, the recombinant construct may contain a promoter upstream of the polynucleotide such that the polynucleotide is under the control of the promoter for expression in the cells or the plant. The promoter should be active or functional in the prokaryotic or eukaryotic cells where the polynucleotide should be expressed. The recombinant construct may further comprise other sequences such as regulatory sequences for expression of the polynucleotide, such as a 3′ UTR (3′ non-translated sequence) or a terminator downstream of the polynucleotide of the invention. Both prokaryotic and eukaryotic expression of polynucleotides is known to the skilled person. The recombinant construct may further comprise other desired genes or gene constructs that are desired to be co-expressed with the polynucleotide of the invention.

In one embodiment, the recombinant construct may comprise a further nucleic acid or gene to be expressed in the prokaryotic or eukaryotic cells. Expression of such further gene may be under the control of the same or a different promoter as the promoter used for expressing the polynucleotide of the invention. The recombinant construct may further comprise a selectable marker gene for selecting cells containing the recombinant construct, e.g. during cloning or when transforming the prokaryotic or eukaryotic cells. This further nucleic acid or gene may encode a CoA ligase that is capable of producing a CoA ester (e.g. those of formulae 1, 3, 5, 7, or 9 below) from the corresponding carboxylic acid and Coenzyme A (CoA). The CoA ligase may be, but is not limited to, a piperoyl-CoA ligase (PipCoA ligase) for producing piperoyl-CoA from piperic acid. The CoA ligase can be cloned in a different vector (transformed into the same cells, with a different selection marker) or on the same vector (like petDuet-1, Novagene) as the polynucleotide of the invention under the control of any promoter, e.g. the T7 promoter, to achieve simultaneous functional expression of two genes, preferentially, but not limited to, the combination of PipCoA ligase with the protein of the first or second general embodiment. Such and similar embodiments allow production and the purification of the produced amides from externally supplied carboxylic acids and the amines in a pro- or eukaryotic host, without the expensive and tedious chemical or otherwise separate enzymatic preparation of the CoA ester from the carboxylic acid, exemplified by Eudes A et al. 2016 for other BAHD-types of enzymes expressed in yeast. Additional genes to enhance the production of the CoA-ester, e.g. by providing anaplerotic reactions for more efficient CoA-ester production, can be transformed separately into the same cells on a different vector with a different selection marker than that of the ligase and synthase.

Coupled reaction from carboxylic acid to amide via the CoA-ester has been successfully achieved in the Examples by a combination of PipCoA ligase and PipSyn2 cloned on a petDuet-1 vector (Novagene) transformed into E. coli SoluBL21 cells (Amsbio), induced by IPTG at an OD of 0.4, grown at 25° C. for 16 hours and fed by adding 1 mM piperic acid and 10 mM piperidine, both dissolved in 25% DMSO in water directly to the medium. The supernatant was collected and extracted with ethyl acetate. The ethyl acetate fraction was concentrated to dryness, re-dissolved in 100% methanol, resulting in production of piperine and piperine isomers, as confirmed by LC-MS.

Herein, the term “gene” means a DNA sequence comprising a region (transcribed region), which can be transcribed into an RNA molecule (e.g. a mRNA) in a cell, operably linked to suitable regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) such as the polynucleotide of the invention, introns, and a 3′-non-translated sequence comprising e.g. transcription termination sites. For bacterial gene expression, several coding sequences may be combined into an operon. Genes and operons and their use for gene expression in different organisms including prokaryotes and eukaryotes are known to the skilled person.

As used herein, the term “promoter” means a transcription promoter, i.e. a nucleic acid (generally DNA) sequence that is capable of controlling (initiating) transcription in a cell or organism where a polynucleotide is to be expressed. For expression in prokaryotic cells such as in Escherichia choli, promoters generally known from prokaryotic gene expression may be used. Promoters for expression of a nucleic acid in yeast are also generally known such as the galactose-inducible promoter used in the examples.

The invention further provides a vector or plasmid comprising a polynucleotide of the invention or the recombinant construct. The vector and plasmid may comprise a selectable marker allowing selecting for the presence of said vector or plasmid in eukaryotic or prokaryotic cells.

### Prokaryotic or Eukaryotic Cell of the Invention

The invention also provides a prokaryotic or eukaryotic cell, or a plant, comprising the polynucleotide, nucleic acid molecule, vector, plasmid, or the recombinant construct of the invention.

The prokaryotic cell may be any prokaryotic cell usable for cloning the recombinant construct, plasmid and/or vector, or prokaryotic cell used for expressing the polynucleotide or nucleic acid molecule of the invention. An example of a usable prokaryotic cell is Escherichia coll. Other examples are Bacillus subtilis, Corynebacterium glutamicum, photosynthetic cyanobacteria such as Synechocystis sp. PCC6803, or bacteria that can grow on substrates such as methanol, e.g. Methylobacterium extorquens, Methylobacillus sp., Methylophyllus sp., Ancylobacter sp., Hyphomicrobium sp., Xanthobacter sp., Thiobacillus sp., Mycobacterium sp., Paracococcus sp., Methylophaga sp. and Acidomonas sp. Methods of transforming a construct or vector into prokaryotic cells are well-known to the skilled person.

Examples of eukaryotic cells, cell lines or strains are yeast cells, cell lines or strains such as of genus Saccharomyces (most preferably Saccharomyces cerevisiae) or Schizosaccharomyces (e.g. Schizosaccharomyces pombe), Yarrowia lipolytica, or Pichia pastoris. The yeast cells or strains may be edible. Other fungi such as molds, e.g. Fusarium fujikuroi could also be used in the invention. Yeast cells are the preferred eukaryotic cells or cell lines for practicing the invention. Yeast cells may be transformed according to generally known methods, cf. Borts, Rhona H. (1996) Yeast protocols: Methods in cell and molecular biology. Methods in Molecular Biology (Book 53) Humana Press. Methods for nucleic acid expression in S. pombe, including vectors, promoters, terminators, transformation methods, integration into a target region e.g. using homologous recombination are known in the prior art, e.g. from EP 2 468 860 A1. Examples of microalgae are microalgae such as Chlamydomonas reinhardtii, various species of Scenedesmus (e.g. S. obliquus, S. vacuolatus, S. rubescens), Chlorella vulgaris or C. kessleri, Haematococcus pluvialis, or Dunaliella salina. Methods for nucleic acid expression in C. reinhardtii and other microalgae are known in the art as reviewed (Scaife M A et al., 2015).

In one embodiment, the prokaryotic or eukaryotic cell or the plant further contains a gene encoding an enzyme that can produce a reactant (or substrate) of the reaction catalysed by a protein of the invention. The reactant producible by the enzyme is the CoA-ester as described herein. Such enzymes and genes encoding them are known in the art as coenzyme A ligases, such as fatty acid-CoA ligases and coumaroyl CoA ligases. These ligases produce a CoA-ester from a carboxylic acid in an ATP-dependent manner. Thus, co-expression of the ligase with the protein of the invention in cells allows a coupled enzymatic production of an amide from a carboxylic acid.

### Production of the Proteins of the Invention

A protein according to the invention may be produced using known methods of protein expression, e.g. in a standard protein expression system. For protein production, a polynucleotide encoding the protein of the invention may be expressed in a suitable host organism. Methods usable for producing and purifying the protein have been described in the prior art and any of such methods may be used. An E. coli expression system as generally known in the art may, for example, be used. A preferred bacterial expression system makes use of the Lemo BL21 (DE3) bacterial expression system used in the examples below, based on a rhamnose-inducible promotor of a 2^{nd} gene (pLysC) on a 2^{nd} plasmid to control protein expression by controlling T7 RNA-Polymerase. This system provides the benefit that the protein of the invention remains soluble in the liquid medium after homogenizing the cells in which it is expressed.

Alternatively, any other prokaryotic protein expression system including Corynebacterium glutamicum, Bacillus subtilis may be used. If a eukaryotic protein expression system is used, one or more introns may be inserted in the coding sequence of the protein to prevent toxicity in the bacterial organism used for cloning. Eukaryotic expression systems include but are not limited to Saccharomyces spec., Pichia pastoris, Baculovirus insect cultures, transient and stable plant transformation systems for Nicotiana benthamina and Arabidopsis thaliana.

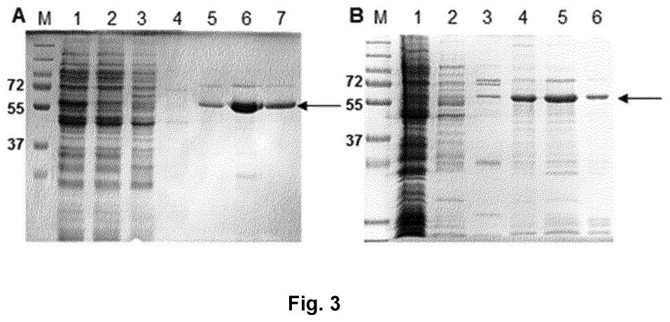

The protein of the invention can be purified to a desired degree according to generally knowns methods (illustrated in Example 2 and FIG. 3). Generally, the protein is extracted into a buffer by breaking cells wherein it is expressed. The buffer preferably has a pH in the range of from 6.0 to 8.0, preferably from 7.0 to 7.8. The buffer may contain 20 to 200 mM, preferably 50 to 150 mM, sodium chloride. In order to better preserve the enzymatic activity of the protein, the buffer may contain from 5 to 30, preferably 10 to 25% by weight glycerol. They buffer may further contain 1-5 mM reducing agent such as dithiothreitol (DTT) or dithioerythritol (DTE), and/or ethylenediamine tetraacetate (EDTA) e.g. 0.1 to 5 mM, preferably 0.5 to 2 mM. After removal of cell debris, generally by centrifugation, the protein can be further purified by conventional methods, such as by column chromatography. A convenient chromatographic method is affinity chromatography, e.g. using an affinity tag on the protein and a column material capable of binding the affinity tag. After rinsing unbound components from the column, the protein can be eluted using a buffer containing a component that competes with the protein for the binding sites on the column, such 100-500 mM imidazole, preferably 200-300 mM imidazole, in the case of a His-tag and a Nickel-NTA-affinity column. Preferably, the elution buffer used contains components as mentioned above such as the glycerol and DTT. After elution from the column, the buffer of the protein solution may be exchanged to remove the component that competes with the protein for the binding sites of the column. Notably, in the case of a His-tagged protein and Nickel-NTA-affinity chromatography, it is preferred to exchange the buffer to remove (“desalt”) imidazole from the buffer of the protein. Preferably, the new buffer contains components as mentioned above such as glycerol and DTT. EDTA may also be contained as described above.

### Process of Producing an Amide

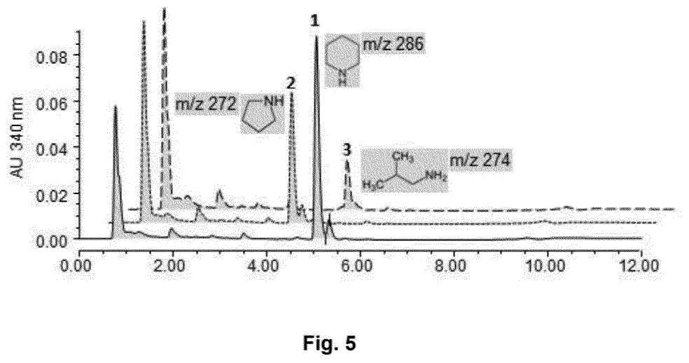

The process of producing an amide according to the invention comprises reacting an acyl-CoA and an amine in the presence of a protein according to the invention. Since the protein of the invention is not limited to piperoyl-CoA and piperidine as substrates, many different amides can be produced. Some examples of products that can be produced using piperoyl-CoA as the CoA-donor and three different amines are shown in FIG. 5. Further examples are given in Example 4. Another example is capsaicin that may be produced from (E)-8-methyl-6-nonenoyl-CoA and vanillylamine, preferably using a protein of the first general embodiment.

The amine is preferably a primary or secondary amine, i.e. a compound having a primary or secondary amino group. The amine may even have two or more primary or secondary amino groups. However, in this case, mixtures of amides may be produced. Therefore, it is preferred that the amine has one primary or one secondary amino group. Other functional groups on the amine generally do not abolish reactivity with the protein of the invention, and the invention is not limited in this regard.

The amine may be a C₁-C₁₄ amine, preferably a C₂-C₁₂ amine, more preferably a C₃-C₁₀ amine, and most preferably a C₅-C₈ amine. The numbers of carbon atoms in the previous sentence refer to the number of all carbon atoms of the amine. In more detail, the primary amine may be a hydrocarbylamine or a heterohydrocarbylamine. The secondary amine may be a di-N-hydrocarbylamine, a N-hydrocarbyl-N-heterohydrocarbyl-amine, or a di-N-heterohydrocarbylamine, such as a di-N—C₁-C₆-hydrocarbylamine, a N—C₁-C₆-hydrocarbyl-N—C₁-C₆-heterohydrocarbyl-amine, or a di-N—C₁-C₆-heterohydrocarbylamine. The heteroatoms of the heterohydrocarbyl moieties are selected from O, N, and S, and are preferably O. There may be from 1 to 3 heteroatoms per heterohydrocarbyl moiety, preferably 1 or 2, most preferably 1. A preferred heterohydrocarbyl group is a heterohydrocarbyl having one oxygen atom. The hydrocarbyl moieties may be linear or branched, or may be cyclic hydrocarbyl moieties, or may comprise linear, branched, and/or cyclic moieties.

Examples of hydrocarbyl groups are methyl, ethyl, propyl, butyl, pentyl, hexyl, cyclohexyl, and phenyl groups. Examples of heterohydrocarbyl groups are methoxy, ethoxy, methylthio, morpholino, and vanillyl groups.

Examples of amines are the following substituted or unsubstituted amines (the carbon atoms of each of the following examples may be, as described above, C₁-C₁₄, preferably C₂-C₁₂, more preferably C₃-C₁₀, and most preferably C₆-C₈): alkylamine, di-N-alkylamine, cycloalkyl-amine, heterocycloalkyl-amine, N-alkyl-N-cycloalkyl-amine, cycloalkenylamine, N-alkyl-N-cycloalkenyl-amine, N-cycloalkylalkylamine, N-alkyl-N-cycloalkylalkylamine, aniline, N-alkyl-aniline, aniline, N-cycloalkyl-aniline, N-phenylalkylamine, N-alkyl-N-phenylalkyl-amine. The carbon atom number ranges including the preferred ones given above preferably also apply to these amines and include carbon atoms of any substituents. Preferably, the amine is selected from substituted or unsubstituted C₁-C₆-alkylamine, di-N—C₁-C₆-alkylamine, C₄-C₇-cycloalkyl-amine, C₃-C₆-heterocycloalkyl-amine, N—C₁-C₆-alkyl-N—C₄-C₇-cycloalkyl-amine, C₄-C₇-cycloalkenylamine, N—C₁-C₆-alkyl-N—C₄-C₇-cycloalkenyl-amine, N—C₄-C₇-cycloalkyl-C₁-C₆-alkylamine, N—C₁-C₆-alkyl-N—C₄-C₇-cycloalkyl-C₁-C₆-alkylamine, aniline, N—C₁-C₆-alkyl-aniline, N—C₄-C₇-cycloalkyl-aniline, N-phenyl-C₁-C₆-alkylamine, N—C₁-C₆-alkyl-N-phenyl-C₁-C₆-alkyl-amine.

In another embodiment, the (secondary) amine is a substituted or unsubstituted cyclic amine. The carbon atom numbers including the preferred ones given above also apply to these amines and include carbon atoms of possible substituents. The cyclic amine group of the unsubstituted cyclic amine or the cyclic amine may be a 4-, 5-, 6, or 7-membered nitrogen-containing ring, preferably a 5- or 6-membered nitrogen-containing ring. The ring may be saturated or unsaturated and may contain an (preferably one) additional heteroatom selected from O, N or S, preferably O. Examples of the cyclic amine are substituted or unsubstituted azetidine, pyrrole, pyrroline, pyrrolidine, imidazole, imidazoline, oxazole, oxazoline, oxazolidine, pyridine, dihydropyridine, piperidine, piperazine, morpholine, and azacycloheptane.

Possible substituents are C₁-C₆-hydrocarbyl or C₁-C₆-heterohydrocarbyl. In more detail, the substituents may be C₁-C₆-alkyl, C₁-C₆-alkoxy, C₃-C₇-cycloalkyl, C₁-C₆-alkoxy, hydroxy, hydroxy-C₁-C₆-alkyl, halo (F, C₁, Br, I), sulfhydryl, oxo, C₃-C₆-aryl, C₃-C₆-heteroaryl (preferably containing one heteroatom selected from O, N, or S, preferably O), or two substituents on adjacent carbon atoms together form a five- or six-membered ring. The five- or six-membered ring may be saturated or unsaturated and may contain a heteroatom selected from O, N, and S, preferably O.

Specific examples of amines are piperidine, pyrrolidine, isobutylamine, benzylamine, 4-aminomethylpiperidine, vanillylamine, and 1,7-diamino-n-heptane.

The acyl-CoA-ester of the invention may be a C₁-C₂₀ acyl-CoA ester, preferably a C₃-C₁₈ acyl-CoA ester, more preferably a C₄-C₁₆ acyl-CoA ester, and more preferably a C₆-C₁₄ ester. As is clear for the skilled person, these carbon atom number ranges relate to the carbon atom numbers of the acyl group. The acyl moiety may be a hydrocarbylcarbonyl or a heterohydrocarbylcarbonyl group, wherein the heterohydrocarbyl group comprises from 1 to 3 heteroatoms selected from O, N, and S, preferably selected from O and S, and even more preferably 1 or 2 heteroatoms selected from O and S, preferably O.

The acyl group may be a substituted or unsubstituted alkylcarbonyl, alkenylcarbonyl such as (E)-8-methyl-6-nonenoyl, alkadienylcarbonyl carbonyl, alkynylcarbonyl, cycloalkylcarbonyl, cycloalkenylcarbonyl, alkylcycloalkylcarbonyl, cycloalkylalkylcarbonyl, cycloalkylalkenylcarbonyl, cycloalkylalkadienylcarbonyl, alkylcycloalkylalkylcarbonyl group, alkylcycloalkylalkenylcarbonyl, alkylcycloalkylalkadienylcarbonyl, arylcarbonyl, alkylarylcarbonyl, arylalkylcarbonyl such as 5-phenylpentanoyl, arylalkenylcarbonyl, arylalkadienylcarbonyl, alkylarylalkylcarbonyl, alkylarylalkenylcarbonyl, or alkylarylalkadienylcarbonyl group. The carbon atom numbers given above for the acyl group of the acyl-CoA esters preferably also apply to these groups. In the case of substituted groups, the carbon atom numbers given above for the acyl groups of the acyl-CoA esters preferably apply to these groups including carbon atoms of substituents. A preferred alkadienyl moiety is a butadienyl moiety.

Possible substituents are the same as those given above in the context of the amine.

In one embodiment, the amine is a C₁-C₁₄ amine and the CoA-ester is a C₁-C₂₀ acyl-CoA ester. In another embodiment, the amine is C₃-C₁₀ amine and the CoA-ester is a C₃-C₁₈ acyl-CoA ester. In another embodiment, the amine is C₅-C₈ amine and the CoA-ester is a C₄-C₁₆ acyl-CoA ester or, preferably, a C₆-C₁₄ acyl-CoA ester.

In another embodiment, the acyl-CoA is a compound of the following general formula (1):

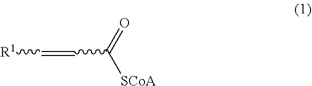

preferably of the following general formula (3):

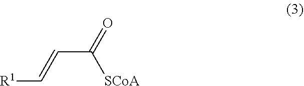

wherein R¹ is a C₁-C₁₇ hydrocarbyl group (preferably a C₁-C₁₅ hydrocarbyl group) or C₂-C₁₆ heterohydrocarbyl group (preferably a C₂-C₁₂ heterohydrocarbyl group, more preferably a C₂-C₁₀ heterohydrocarbyl group), and SCoA is a CoA group (reciting the S merely highlights the terminal sulfur atom of the CoA group). As above, the heterohydrocarbyl group contains from 1 to 3 heteroatoms selected from O, N, and S, preferably selected from O and S, and even more preferably 1 or 2 heteroatoms selected from O and S, preferably O. As is generally known, the wiggly line in the compound of formula (1) and in the formulae below is a single bond and indicates that the stereochemistry of the double bond is not defined.

Products of the reaction involving compounds of formulae (1) and (3) are the compounds of the following formulae (2) and (4), respectively:

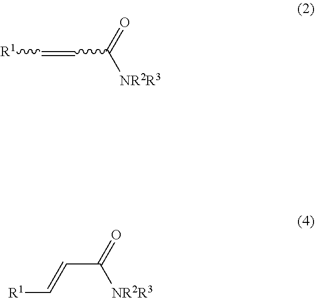

wherein R¹ is as defined above, and NR²R³ is the amino group derived from the amine used in the invention and described above. In more detail, R² is a hydrogen atom, a hydrocarbyl group as described above, or a heterohydrocarbyl group as described above; R³ is a hydrocarbyl group as described above or a heterohydrocarbyl group as described above; or R² and R³ together form with the N-atom to which they are bound an optionally substituted 4-, 5-, 6, or 7-membered nitrogen-containing ring, preferably a 5- or 6-membered nitrogen-containing ring, as described above. The ring may be saturated or unsaturated and may contain an (preferably one) additional heteroatom selected from O, N or S, preferably O. Optional substituents are as defined above. The group NR²R³ is, including optional substituents, preferably a C₁-C₁₄—NR²R³-group, more preferably a C₂-C₁₂—NR²R³-group, more preferably a C₃-C₁₀—NR²R³-group, and most preferably a C₅-C₈—NR²R³-group.

Examples of R¹ are C₁-C₁₇ substituted or unsubstituted alkyl, cycloalkyl, alkylcycloalkyl, cycloalkylalkyl, alkylcycloalkylalkyl group, aryl, alkylaryl, arylalkyl, alkylarylalkyl group. Possible substituents are those given above.

In a more preferred embodiment, the acyl-CoA is a compound of the following general formula (5):

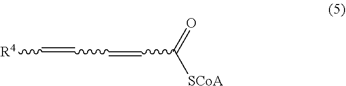

preferably the acyl-CoA is a compound of the following general formula (7):

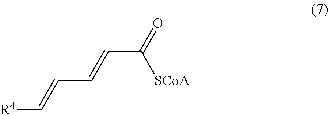

wherein R⁴ is a C₁-C₁₅ hydrocarbyl group (preferably a C₂-C₁₀ hydrocarbyl group, more preferably a C₂-C₈ hydrocarbyl group) or C₂-C₁₄ heterohydrocarbyl group (preferably a C₂-C₁₀ heterohydrocarbyl group, more preferably a C₂-C₈ heterohydrocarbyl group). As above, the heterohydrocarbyl group contains from 1 to 3 heteroatoms selected from 0, N, and S, preferably selected from O and S, and even more preferably 1 or 2 heteroatoms selected from O and S, preferably O. SCoA is a CoA group.

Products of the reaction involving compounds of formula (5) and (7) are the compounds of the following formulae (6) and (8), respectively, (wherein R² and R³ as defined above):

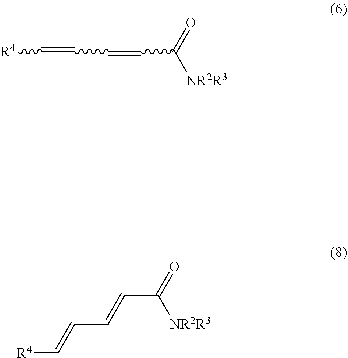

R⁴ is as described above. Examples of R⁴ are C₁-C₁₅ substituted or unsubstituted alkyl, cycloalkyl, alkylcycloalkyl, cycloalkylalkyl, alkylcycloalkylalkyl group, aryl, alkylaryl, alkenylaryl, arylalkyl, alkylarylalkyl, and alkenylarylalkyl groups. Possible substituents are those given above. R² and R³ are as defined above.

In a more preferred embodiment, the acyl-CoA is a compound of the following general formula (9):

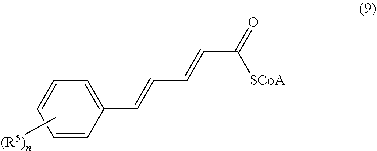

wherein n is an integer of from 1 to 4, preferably 1 or 2, and multiple groups R⁵ may be the same or different, and R⁵ is selected from C₁-C₆ alkyl, C₂-C₆ alkenyl, C₂-C₆ alkynyl, C₁-C₆ alkoxy, hydroxy, or hydroxy C₁-C₆ alkyl, and sulfhydryl, or two R⁵ at adjacent carbon atoms of the phenyl ring together form a five- or six-membered ring, such as a 3,4-methylene dioxy group. SCoA is a CoA group.

Products of the reaction involving compounds of formula (9) are compounds of the following formula (10):

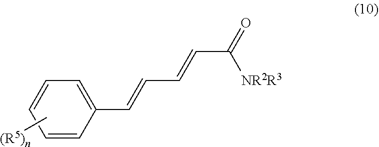

R⁵ is as described above. Also, R² and R³ are as defined above.

In preferred specific embodiments, the acyl-CoA is piperoyl-CoA or 3,4-methylenedioxy cinnamoyl-CoA and the primary or secondary amine is selected from the group consisting of piperidine, pyrrolidine, isobutylamine, benzylamine, 4-aminomethylpiperidine, vanillylamine, and 1,7-diamino-n-heptane, preferably piperidine. In another preferred embodiment, the acyl-CoA is (E)-8-methyl-6-nonenoyl-CoA, the amine is vanillylamine, and the amide product is capsaicin.

The method of the invention may be conducted in a cell-free system or in the presence of, or in, prokaryotic or eukaryotic cells (as described above) expressing the protein of the invention.

In a cell-free system, the method of producing an amide from an acyl-CoA and an amine is generally conducted in an aqueous medium, preferably a buffered aqueous medium. The pH of the medium may be between pH 6.0 and 9.0, preferably between pH 7.0 and 8.5. Generally known buffer substances that buffer at the selected pH may be used, such as Tris. The aqueous medium may be further supplied by a water-soluble polyol such as ethylene glycol or glycerol in a concentration of from 1 to 20 weight-%, preferably from 5 to 15 weight-%. Glycerol is preferred as the polyol.

The buffered solution in the cell-free system also contains a reducing agent, preferentially 1-2 mM DTT to prevent dimerization and oxidation of the enzymes, specifically during longer incubation times. The term “longer” refers to a time frame longer than 5 minutes

The method may be carried out in a method (e.g. a batch method), wherein the protein of the invention is mixed with the aqueous medium containing the acyl-CoA and the amine. The starting concentrations of the acyl-CoA may be from 10 μM-50 mM, preferably from 50 μM-20 mM, more preferably 100 μM-5 mM. The starting concentrations of the amine may be from 100 μM to 100 mM amine, preferably 500 μM to 50 mM, more preferably from 2 to 20 mM.

The protein is added in an amount to give a desired turnover of the substrates to product and is thus not particularly limited. Reactions may contain the protein in a concentration range from 1 μg/ml to 1 mg/ml, preferably from 5 μg/ml to 500 μg/ml, more preferably from 10 μg/ml to 200 μg/ml.

The amide product may be isolated from the aqueous medium as follows. For example, protein components of the reaction system may be precipitated e.g. using trichloroacetic acid, or formic acid together with acetonitrile or ethanol in a 50 to 50 ratio to prevent simultaneous precipitation of the hydrophobic products. This Stop-mix of organic solvent and acid is premade and added to the reaction system and mixed. The protein is removed e.g. by centrifugation or filtration. The supernatant contains the amide reaction product that may be purified by liquid chromatography (LC) as described in the experimental section.

A standard analytical procedure to isolate the amide can be achieved by preparative HPLC, where the reaction mix is applied to a C₁₈ or C₈ reversed phase-(RP) column (available from several suppliers, e.g. Macherey and Nagel, Waters, or Agilent) and the products are separated from buffer, residual substrates, and enzyme by a gradient of increased organic solvent, either methanol or acetonitrile, while individual fractions are collected. The product isolated in one such fraction is concentrated preferentially by freeze-drying, but evaporation of the solvent to dryness may also be possible.

An alternative suitable approach for larger scale production of a desired amide is by standard solid phase extraction cartridges or columns on a reversed phase-C-8 or C-18 matrix or a silica matrix, depending on the product and provided by several manufacturers (Waters, Agilent, Merck). In this case the supernatant of the said protein reaction may be applied in a single batch to the matrix, more hydrophilic substrates elute and the product, such as piperoylpyrrolidine, is batch-eluted with the desired concentration of an organic solvent, preferentially 80-100% methanol or acetonitrile and concentrated as said, by evaporation of the organic solvent, alternatively by adding water to 50% final concentration and subsequent freeze-drying. More precise elution conditions are dependent on the matrix and the properties of the amide produced and require prior testing. Specific conditions need to be optimized for individual products, specifically in preparative large scale processes.

The acyl-CoA for use in the process of the invention may be generated chemically from the coenzyme A and the corresponding carboxylic acid. The carboxylic acid is first activated e.g. by N-hydroxysuccinimide/carbodiimide-based activation of the carboxylic acid, followed by reaction with coenzyme A, which is an established procedure, see Stöckigt and Zenk, 1975.

It is also possible to generate the CoA-ester in situ in an enzymatic reaction from a carboxylic acid and coenzyme A in the presence of a CoA-ligase and a suitable energy source such as ATP. One example is PipCoA-ligase which was isolated from black pepper (Piper nigrum) and can produce piperoyl CoA and 5-phenylpentanoyl CoA from the corresponding acids. Both compounds can serve as CoA-donors in the in vitro reactions using the proteins of the inventions (see Table 1). Individual CoA-esters can be purified by chromatography on silica gel and/or solid phase extraction cartridges.

The invention also provides a process of producing an amide from a carboxylic acid and an amine that is a primary or secondary amine, comprising incubating the carboxylic acid and the amine in the presence of a CoA ligase, coenzyme A, and a protein according to the invention, optionally followed by isolating the amide produced. The amine may be as defined above, and the carboxylic acid may be a compound having the CoA moiety of any of the CoA-esters defined above replaced by a hydroxyl moiety.

The process of the invention is preferably carried out in the presence of, or in, prokaryotic or eukaryotic cells expressing the protein of the invention. The cells may further express a CoA-ligase for generating the acyl-CoA in said cells or in the reaction medium. The process of the invention may be carried within prokaryotic or eukaryotic cells. Alternatively, the protein of the invention (and optionally further enzymes required for the process) may be expressed with a tag that targets the protein to the secretory pathway to secret the protein into the medium. In this case, the process can be carried out in the medium in the presence of the cells that provide the protein(s).

The cells may be cultured in a suitable culture medium to allow the process of the invention to take place. Suitable culture media for various cells are known in the art. If the process is performed in eukaryotic cells, the cells may be a transgenic and contain the polynucleotide of the invention stably and expressibly incorporated in a chromosome. The eukaryotic cells may further contain, as the case requires, any of the other genes mentioned above. Usable cells are those listed above.

After the product has been produced, the product may be isolated and purified. If the product is secreted, it can be purified by solid phase extraction on a suitable matrix, reversed phase or silica, after the cells have been separated by filtration or by centrifugation. Isolation of the product may alternatively be performed from the cells homogenizing the cells containing the product, for example in the presence of an aqueous buffer. Again, insoluble debris or cells may be removed. The amide product may be purified from the aqueous buffer by liquid chromatography (LS) or solid phase extraction as done in the experimental section.

### EXAMPLES

### Example 1: Identification of Candidate Genes, Characterization of the Protein Sequence of PipSynthase1 and 2

To identify piperine synthase and piperamide related genes we monitored piperine formation during fruit development of P. nigrum in greenhouse grown black pepper plants over several months. Drupes of several plants were marked and piperine amounts were quantified by HPLC-MS and UV-detection respectively, based on a standard of commercially available piperine. From this time course, it was evident that in our greenhouse grown plants piperine accumulation started after a lag-phase of roughly 20 days after anthesis and peaked approximately three months after anthesis at levels of 2.5% piperine calculated per fresh weight. No significant increase was observed during later stages of fruit development. Therefore, our focus turned to a time frame between 20-50 days post anthesis, a few days before the maximum slope of piperine increase was observed. For the differential RNA-Seq approach two time periods were selected, a period between 20-30 day post anthesis, where piperine formation just started and a 2^{nd} period between 40-50 days post anthesis based on the assumption that specific transcript levels preceded accumulation of biosynthetic product formation. Spadices with flowers as well as young leaves served as additional negative controls in the differential RNA-Seq approach. In both organs, no or very low piperine signals were detected by LC-MS. From the differential screening, it was evident, that individual organs show unique transcript profiles and contain numerous acyltransferase sequence candidates.

Candidate gene sequences were selected based on i) relative transcript abundance in young fruits of black pepper plants (Piper nigrum) in comparison to leaves and flowers in an RNA-seq experiment and ii) sequence identity to putative BAHD-signature sequences in various databases. This RNA-seq data set was based on plants and plant tissue grown at the IPB greenhouse and has been uniquely designed and developed at the Leibniz Institute of Plant Biochemistry and was a requirement to identify the nucleotide and protein sequences of PipSyn1 and PipSyn2. Full length sequences of the candidate genes were obtained from assembled sequence contigs. One candidate gene, PipSynthase 1 (also PipSyn1), revealed an open reading frame of 1383 nucleotides (SEQ ID NO: 1) and the other candidate gene, PipSynthase2 (also PipSyn2), an open reading frame of 1386 nucleotides (SEQ ID NO: 3). Sequences were assembled from fragments obtained by Illumina High Throughput Sequencing of the transcriptome of black pepper according to Haas B J et al., 2013.

The nucleotide sequence of SEQ ID NO: 1 (PipSynthase1) was in silico-translated into the amino acid sequence of SEQ ID NO: 2 and the nucleotide sequence of SEQ ID NO: 3 (PipSynthase2) was in silico-translated to the amino acid sequence of SEQ ID NO: 4. The amino acid sequences of SEQ ID NO: 2 and 4 were then BLASTed against protein databases to find similar amino acid sequences. The identified protein sequences of SEQ ID NO: 2 and 4 were found to have some similarity to BAHD-type transferases and the results are displayed in the dendrogram in FIG. 1. Both PipSynthase1 (SEQ ID NO: 2) and PipSynthase2 (SEQ ID NO: 4) clustered among, but were distinct from, benzoyl-benzoate transferases (see circle in FIG. 1). From the benzoyl-benzoate transferases displayed in FIG. 1, only the BAHD-type transferase of Clarkia breweri has been characterized so far. Other BAHD-type transferases from FIG. 1, either functionally explored like the vinorine synthase or only predicted based on sequence information like the capsaicin synthase (PUN1) appear distantly related to SEQ ID NO: 2 and SEQ ID NO: 4, both in terms of sequence similarity (<20%) and catalytic function.

Both SEQ ID NO: 2 and SEQ ID NO: 4 contain an HXXXD-motif consistent with the family of BAHD-type acyltransferases. The histidine is required for the abstraction of a proton as the initial step of the reaction mechanism and considered to be characteristic for all BAHD-like transferases. In addition, several other structural motifs of BAHD-type transferases are present. The overall amino acid sequence identity to the closest sequence which is annotated as a benzoyl-benzoate transferase from Clarkia brewerii (Onagraceae) was about 42%, whereas the sequence identity between SEQ ID NO: 2 and SEQ ID NO: 4 is 62% at the amino acid level and 69% at the nucleotide sequence level. Other similar proteins of the BAHD-family in that sub-cluster, which are not functionally characterized, show sequence identities of 40%, and the sequence identities in other sub-clusters are between 15-25% (FIG. 1 and FIG. 2). SEQ ID NO: 2 and 4 contain a structure motif outside the catalytic center, comprising the amino acid stretch DWGWG instead of DFGWG, which is present in all other amino acid sequences of proteins depicted in FIG. 1 and FIG. 2 except PipSynthase 1 and 2.

### Example 2: Protein Expression of PipSynthase1 and 2

Full length nucleotide sequences of SEQ ID NO:1 and SEQ ID NO: 3 were amplified from cDNA extracted from young black pepper fruits by Phusion (Thermofischer Scientific) polymerase using the primers:

forward:
(SEQ ID NO: 8)
5′ CATCATATGGCTTCTTCTCAGCTCGAATTC 3′
reverse:
(SEQ ID NO: 9)
5′ CATGGATCCTTACATGCGGGACATGTACCCATG 3′
and SEQ ID 3:
forward
(SEQ ID NO: 10)
5′ CATCATATGGCGCCTTCTTCTCAACTTG 3′
reverse
(SEQ ID NO: 11)
5′ CATGGATCCTTACATGCGGGAAAGGTATCCATC 3′

subsequently A-tailed by Flexi DNA-polymerase (Promega) to add A-overhangs and then cloned into the pGem-T easy cloning vector (Promega). This vector was then used to transform E. coli DH10B blue cells. The transformed cells were plated on liquid broth agar, LB, (Duchefa) supplemented with 50 μg/ml ampicillin and grown over night at 37° C. For each construct, a single colony was picked to inoculate into liquid media culture, LB high salt (Duchefa), supplied with 50 μg ml⁻¹ ampicillin and grown over night at 37° C. After harvest and plasmid isolation, the cloned sequences were verified by sequencing. The verified sequences were excised by the restriction enzymes NdeI and BamHI according to the manufacturer's protocol and cloned into NdeI/BamHI restriction sites of the protein expression vector pet16b (Novagen), followed by ligation with T4 DNA-ligase (Thermofischer Scientific).

The ligated expression vector was then used to transform Lemo BL21 (DE3) cells (New England Biolabs) according to the manufacturer's protocol. These cells served as the protein expression system. The cells were plated on LB agar supplemented with 50 μg/ml ampicillin and grown over night at 37° C. A single bacterial colony for each of the expression constructs comprising SEQ ID NO: 1 or SEQ ID NO: 3 was picked to inoculate a 25 ml liquid pre-culture in LB medium containing 50 μg/ml ampicillin and grown overnight at 30° C. An 8 ml aliquot of the pre-culture was then used to inoculate the 400 ml-liquid main-culture containing 50 μg/ml ampicillin and 0.4 mM rhamnose. At a cell density of 0.6 OD₆₀₀, recombinant protein expression was induced by addition of 1 mM IPTG, and the culture was grown for 18 to 22 h at 25° C.

The Lemo BL21 (DE3) bacterial expression system used herein is based on a rhamnose-inducible promotor of a 2^{nd} gene (pLysC) on a 2^{nd} plasmid to control protein expression by controlling T7 RNA-Polymerase, which provides the transcriptional control of the sequence to be expressed. This system allowed tunable expression of proteins encoded by SEQ ID NO: 1 and SEQ NO: 3 at lower amounts than conventional expression system, like BL21DE cells (Novagen, NEB) or M15Rep4 cells (Qiagen), but provided the benefit that large parts of the recombinant proteins can be extracted as soluble recombinant proteins after bacteria are collected from liquid culture and are extracted by sonication (info by the manufacturer and verified by our experimental approach).

After cultivation, the cells of the Lemo BL21 (DE3) expression system were harvested by centrifugation at 4000 g for 10 min at 4° C. Pellets were resolved in 50 ml volume buffer (20 mM Tris-HCl, pH 7.5, containing 100 mM NaCl and 15% glycerol) and disrupted by sonification on ice. The homogenate was centrifuged at 10,000 g for 10 min at 4° C. to pellet the debris. The supernatant was tested for PipSynthase activity by a standard enzyme activity test with 20 μg of crude protein, instead of 2-5 μg of purified protein, and product formation was analyzed by LC-MS as reported and illustrated in Example. 3. The supernatant was then applied to a 1 ml prepacked Ni-NTA column (Macherey-Nagel). This is possible, since the pET16b vector contains an N-terminal protease cleavable His-Tag cloned in frame with the protein according to SEQ ID NO: 1 and 2. His-tagged PipSynthases were eluted with 300 mM imidazole, 20 mM Tris-HCl, pH 7.5, 100 mM NaCl and 15% glycerol. The purified proteins were then desalted into 30 mM Tris-HCl pH 8.0, 1 mM DTT, 10% glycerol and stored at −80° C. and remained stable over several freeze/thaw cycles. The purified protein of PipSynthase1 and 2 was visualized on SDS-PAGE gels (FIG. 3).

### Example 3: Testing Enzyme Activity of PipSynthase1 and 2

Standard assays are routine for a person of average skill in the art.

Enzyme activity test with regard to piperine formation in case of PipSynthase 2.

A standard enzyme assay contains:

### 10 μl 120 mM Tris/HCl at pH 8.0

10 μl enzyme solution in 30 mM Tris/HCl buffer pH 8.0 (10% glycerol, 1 mM DTT)

1 μg-5 μg protein, amount determined according to Bradford

10 μl piperidine, 100 mM (in water)
10 μl 5 mM DTT in water
10 μl piperoyl-CoA (PipCoA) 2.5 mM in 25% DMSO/75% water
All reagents (except PipCoA) were mixed in Eppendorf tubes and incubated in a standard (Eppendorf) incubator set at 30° C. Reagents were pre-equilibrated for 1 min to this temperature.
Assay start: addition of 10 μl PipCoA 2.5 mM in 25% DMSO/75% water to a final concentration of 500 μM PipCoA.
Assay stop after 5 min addition of 10 μl stop solution such as 1% formic acid/50% acetonitrile

Mix by vortex, incubate on ice for 5 minutes and centrifugation for 1 min at 20,000 g to precipitate any debris or insoluble material e.g. residual protein.

Remove 50 μl of supernatant for analysis of products (amides) by LC-MS.
Injected to an RP-HPLC column are 10 μl (out of 50 μl).
Product formation is recorded via UV/PDA (photodiode array) detector and LC-MS and simultaneously.
Identification of piperine was achieved by use of a commercial standard and by LC-MS detection of calculated product masses at m/z 286 and UV-detection at 340 nm.
Quantification of product formation was achieved by a standard graph of piperine from 0.1-100 μM (commercial standard, Sigma) and recorded and calculated intensities of mass (peak area) and/or UV-peak area.
Identification and separation of piperine was according to a corresponding standard and a mass signal of m/z 286.1 in pos. mode and its corresponding absorbance at λₘₐₓ-340 nm.

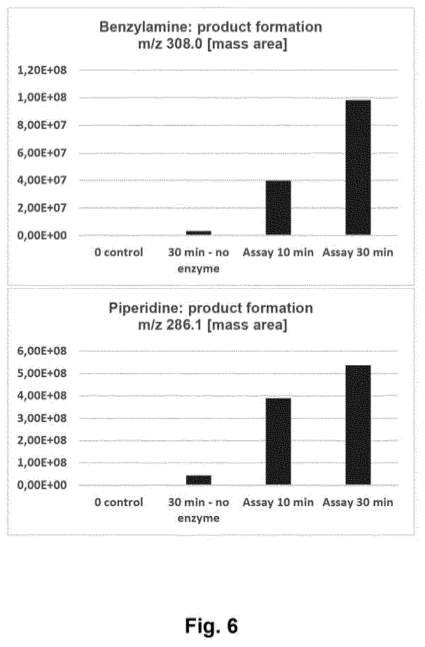

Quantification was performed based on a commercially available piperine standard. The peak area of the peak due to piperine in the LC trace was determined. Peaks due to possible isomerization products of piperine were neglected for the determination of enzyme activity of piperine formation. Product formation was observed without protein and under otherwise identical conditions (FIG. 6). Before calculating enzyme activity in terms of nkat (nmole of piperine formed per second), the amount of non-enzymatically formed piperine is subtracted from the amount determined in the presence of protein.

Protein amounts were determined photometrically by the Bradford assay of combining a defined volume, preferentially 10 μl of the (partly) purified PipSyn1 and PipSyn2 enzyme and 990 μl of Bradford reagent, and calculating the resulting absorbance at λ=595 nm based on a standard curve made with a serial dilution of commercially available Bovine Serum Albumin fraction V in water.

### Example 4: Testing Substrate Promiscuity

This Example provides in Table 1 substrates which were tested for enzyme activity with both enzymes. Activity data were calculated from the UV-(PDA) area in the range of 320-370 nm. The data are supported by LC-MS measurements of corresponding m/z values recorded in positive ionization mode and based on calculated expected masses. Mass accuracy of Waters QDA-detector is +/−0.2 Dalton. Contrary to the procedure of Example 3, for the determination of the activity of the reaction of piperoyl-CoA and piperidine as substrates, the sum of all peaks in the LC trace due to species having calculated m/z values of the expected products was used for quantification of product, assuming the same ionization efficiency in positive ionization mode as in case of piperine for all molecule species.

TABLE 1
Combinatorial table of the substrates, CoA-esters and amines, and relative
activities. Activities were calculated based on and were tested with our in vitro assay
system as described in Example 3. Identification was performed via LC-UV and LC-MS
detection. Quantification was performed by UV-detection in case of piperoyl CoA, 3,4-
methylenedioxycinnamoyl CoA and benzoyl CoA, where products can be easily identified
by UV-absorbance. Quantification was performed by mass detection of product formation
in case of hexanoyl CoA and myristoyl CoA, compared to piperoyl CoA. Due to the
absence of commercially available amide standards except piperine, in case of aromatic
compounds the corresponding UV-signal supported identification by mass spectrometry,
whereas in case of hexanoyl and myristoylamide formation, identification was solely based
on calculated mass predictions (detection limits of the QDA detector: +/−0.2 dalton).

| | | | 3,4- | | | |
|---|---|---|---|---|---|---|
| | | | methylene- | | | |
| 2 nkat/mg | | | dioxy- | | | |
| recombinant PipSyn2 | | piperoyl- | cinnamoyl- | benzoyl- | hexan- | myristoyl- |
| or piperidine = 100% | Enzyme | CoA | CoA | CoA | oyl-CoA | CoA |
| piperidine | PipSyn2 | 100 | 38 | n.d. | 12 | 10 |
| | PipSyn1 | 117 | 184 | 7 | 23 | 10 |
| pyrrolidine | PipSyn2 | 34 | 3 | n.d. | 60 | <1 |
| | PipSyn1 | 77 | 186 | 1 | 300 | 3 |
| isobutylamine | PipSyn2 | 16 | 2 | n.d. | n.d. | 15 |
| | PipSyn1 | 66 | 173 | 6 | n.d. | 20 |
| 5-aminomethylindole | PipSyn2 | n.d. | n.d. | n.d. | n.t. | n.t. |
| | PipSyn1 | n.d. | n.d. | n.d. | n.t. | n.t. |
| benzylamine | PipSyn2 | 12 | 28 | n.d. | n.t. | n.t. |
| | PipSyn1 | 117 | 137 | 8 | n.t. | n.t. |
| 4-aminomethylpiperidine* | PipSyn2 | 36 | 7 | n.d. | n.t. | n.t. |
| | PipSyn1 | 17 | 6 | n.d. | n.t. | n.t. |
| vanillylamine | PipSyn2 | n.d. | n.d. | n.d. | <1 | n.d. |
| | PipSyn1 | 2 | 10 | n.d. | 2 | n.d. |
| 1,7-diaminoheptane | PipSyn2 | 2 | n.d. | n.d. | n.t. | n.t. |
| | PipSyn1 | 26 | 104 | 2 | n.t. | n.t. |
| dopamine | PipSyn2 | n.d. | n.d. | n.d. | n.t. | n.t. |
| | PipSyn1 | 2 | 7 | n.d. | n.t. | n.t. |

n.d. not detected;
*only one out of two amino groups reacted.,
n.t., not tested

### Example 5

Production of piperine in a fermentation process using E. coli was achieved by combining the sequences of said PipCoA ligase and said PipSyn2, both cloned on a petDuet-1 vector (Novagene) transformed into E. coli SoluBL21 cells (Amsbio), induced by IPTG at an OD of 0.4, grown at 25° C. for 16 hours and fed with substrates by adding 1 mM piperic acid and 10 mM piperidine, both dissolved in 25% DMSO (final concentrations) directly to 1 ml of the said E. coli cell suspension. After 30 min, the cells were centrifuged, the supernatant was collected, and extracted with 100% ethyl acetate. The ethyl acetate fraction was concentrated to dryness, redissolved in 100% methanol and checked by LC-MS, as said, resulting in the production of piperine and piperine isomers.

### Example 6: Process of Producing an Amide In Vitro and In Vivo

A standard analytical procedure to isolate the amide in small quantities from enzyme assays in a cell free system can be achieved by preparative HPLC, where the reaction mix is applied to a C₁₈ or C₈ reversed phase-(RP) column (available from several suppliers, e.g. Macherey and Nagel, Waters, or Agilent) and the products are separated from buffer, residual substrates and enzyme by a gradient of increased organic solvent, either methanol or acetonitrile, while individual fractions are collected. The product isolated in one such fraction is concentrated by freeze drying and evaporation of the solvent to dryness. This is a routine procedure and can be achieved by any researcher provided with the corresponding LC and freeze drying equipment.

An alternative approach for larger scale production of the desired amide is by standard solid phase extraction on a RP-matrix (or a silica matrix), depending on the product with the solid phase provided by several manufacturers (Waters, Agilent, Merck), where the supernatant of the protein reaction is applied in a single batch to the solid phase matrix where hydrophilic impurities are washed in aqueous solutions from the matrix of increasing hydrophobicity, usually from 50-100% methanol or acetonitrile and concentrated, preferentially by freeze drying. Exact elution conditions are dependent on the matrix and the properties of the amide and can be determined by testing. Specific conditions are preferably optimized for individual products, specifically in preparative large scale processes.

### REFERENCES

Bauer A et al. (2019) A short, efficient, and stereoselective synthesis of piperine and its analogues. Synlett, 30, pp. 413-16. doi:10.1055/s-0037-1611652.
Bradford M M (1976) A rapid and sensitive method for the quantitation of microgram quantities of protein utilizing the principle of protein-dye binding. Analytical Biochem. 72, 248-254. doi.org/10.1016/0003-2697(76)90527-3
Eudes A et al. (2016) Exploiting members of the BAHD acyltransferase family to synthesize multiple hydroxycinnamate and benzoyl conjugates in yeast. Microb. Cell Fac. 15, pp. 198-213. doi.org/10.1186/s12934-016-0593-5
Fregnan, A M et al. (2017) Synthesis of piplartine analogs and preliminary findings on structure-antimicrobial activity relationship. Medicinal Chem. Res. 26, pp. 603-614. doi:10.1007/s00044-016-1774-9.
Geisler J G and Gross G G (1990) The biosynthesis of piperine in Piper nigrum. Phytochemistry 29 pp. 489-92. doi:10.1016/0031-9422(90)85102-L.
Haas B J et al. (2013) De novo transcript sequence reconstruction from RNA-seq using the Trinity platform for reference generation and analysis. Nature Prot. 8, pp. 1494-1512. doi: 10.1038/nprot.2013.084
Leistner E and Spenser ID (1973) Biosynthesis of the piperidine nucleus. Incorporation of chirally labeled [1-³H] cadaverine. J. Am. Chem. Soc. 95, pp. 4715-4725. doi:10.1021/ja00795a041.
Li H et al. (2018) “Capsaicin and piperine can overcome multidrug resistance in cancer cells to doxorubicin”. Molecules, 23, p. 557. doi:10.3390/molecules23030557.
McNamara, F N et al. (2005) Effects of Piperine, the pungent component of black pepper, at the human vanilloid receptor (TRPV1). Brit. J. Pharmacol. 144, pp. 781-790. Wiley Online Library, doi:10.1038/sj.bjp.0706040.
Mih\check{a}il\check{a}, B al. (2019) New insights in vitiligo treatments using bioactive compounds from Piper nigrum. Exp. Therapeut. Med., 17, pp. 1039-1044 doi:10.3892/etm.2018.6977.
Navickiene D H M et al. (2007) Toxicity of extracts and isobutyl amides from Piper tuberculatum: Potent compounds with potential for the control of the velvet bean caterpillar Anticarsia gemmatalis. Pest Management Sci. 63, pp. 399-403. DOI: 10.1002/ps.1340
Scaife M A, et al. (2015). Establishing Chlamydomonas reinardtii as a biotechology industrial host. Plant J. 82, pp. 532-546. doi: 10.1111/tpj.12781.
Suzuki T and Yamato S (2018) Mode of action of piperovatine, an insecticidal piperamide isolated from Piper piscatorum (Piperaceae), against voltage-gated sodium channels”. NeuroToxicol. 69, pp. 288-295. doi:10.1016/j.neuro.2018.07.021.
Stöckigt J and Zenk M H (1975) Chemical syntheses and properties of hydroxycinnamoyl-coenzyme A derivatives. Z. Naturforsch. C30, pp. 352-358. doi.org/10.1515/znc-1975-5-609.
Ternes W and Krause E L (2002) Characterization and determination of piperine and piperine isomers in eggs. Anal. Bioanal. Chem. 374, pp. 155-160. doi.org/10.1007/s00216-002-1416-6.
Wiemann J et al. (2017) Piperlongumine B and analogs are promising and selective inhibitors for acetylcholinesterase. Eur. J. Med. Chem. 139, pp. 222-231. doi.org/10.1016/j.ejmech.2017.07.081
Xie Z et al. (2019) Alkaloids from Piper nigrum synergistically enhanced the effect of paclitaxel against paclitaxel-resistant cervical cancer cells through the downregulation of Mcl-1”. J. Agric. Food Chem. 67, pp. 5159-68. doi:10.1021/acs.jafc.9b01320.

Nucleotide and amino acid sequences
SEQ ID NO: 1 DNA sequence PipSynthase 1 (1380 nucleotides; TAA-stop codon
not included)
ATGGCTTCTTCTCAGCTCGAATTCAATGTGGAGAGGAAGCAACCGGAGCTTCTCGGCCCGGCAGAACCAACTCCCTACG
AGTTGAAGGAGCTGTCGGACATCGACGACCAAGACGGTGTCCGCCTCTTTTTGACTGCCATTTTCATATACCCCCCACCA
ACCAAGACATCCATGCCCACCCGCAAAACCGACCCAGCTAGCGACATCCGCCGGGGCCTATCCAAAGCTATGGTCTACT
ACTACCCTTTTGCCGGCCGTATAAGAGAAGGCCCCAACAGGAAGCTATCCGTTGACTGCACCGGCGAGGGAATCGTGTT
CTGCGAGGCCGACGCCGACATTCGGCTAGATGGATTGGGTGACGTCGAGGTGCTCCGTCCGCCCTACCCATTCATAGA
CAAGATGACGCTGGGAGAGGGGAGTGCCATCCTCGGCGCACCCTTGGTGTACGTGCAGGTGACCCGCTTTGCCTGCGG
TGGGTTCATCATCACCGGACGTTTCAACCATGTCATGGCCGATGCGCCGGGGTTTACCATGTTTATGAAGGCCGCGGCC
GACCTCGCACGCGGAGCCACCGTCCCGATGCCGCTACCGGTGTGGGAGCGGGAGCGTTACCGGTCGCGAGTGCCGCC
CCGGGTCACCTTCGCCCACCATGAATACATGCACGTCGACGATCCCCCGCCCAGACCTACGAGCGAGCCATGGTCATTG
CACTCCGCCTTCTTCACCAAGGCTGACGTCGCCACGCTCCGGGCACAGCTGCCGGCCGACCTTCGGAAAGCGGCAACC
TCCTTTGATATCATCACCGCCTGCATGTGGCGGTGCCGGGTGTCGGCACTCCAGTACGGGCCGGACGAGGTGGTTCGA
CTGATCGTCGCCGTCAACTCCCGGACCAAGTTCGACCCTCCTCTAACAGGGTACTACGGTAACGGCCTCATGCTCCCGG
CCGCCGTGACTGAAGCCGGGAAGCTAGTCGGGAGCGACCTTGGGTACGCGGTGGAGCTGGTGAGGGAGGCCAAGGGG
AAGGTGACGGAGGAGTACGTGCGGTCGGCGGCGGACTTCTTGGTGCTCAATGGGAGGGTCCATTTCGTGGTGAGCAAT
ACGTTCTTGGTGTCGGATCTTCGGCGGTTGATTGATTTGGCGAATATGGATTGGGGATGGGGGAAGGCGGTGTCCGGTG
GGCCGGTGGATGTAGGGGAGAATGTGATAAGCTTCTTGGCGACGTCGAAGAACAGTGCAGGGGAGGAAGGAGCGGTG
GTTCCTTTCTGCTTGCCGGATTCTGCCCTGGGAAGGTTCACGTCGGAGGTTAAGAAGCTGGTTTGTTTTCGTCCGTTGGA
GAACGCGGCGGCGTCGAATCCAGATCATGGGTACATGTCCCGCATG
SEQ ID NO: 2 Amino acid sequence PipSynthase1 (460 amino acids)
MASSQLEFNVERKQPELLGPAEPTPYELKELSDIDDQDGVRLFLTAIFIYPPPTKTSMPTRKTDPASDIRRGLSKAMVYYYPFAG
RIREGPNRKLSVDCTGEGIVFCEADADIRLDGLGDVEVLRPPYPFIDKMTLGEGSAILGAPLVYVQVTRFACGGFIITGRFNHVM
ADAPGFTMFMKAAADLARGATVPMPLPVWERERYRSRVPPRVTFAHHEYMHVDDPPPRPTSEPWSLHSAFFTKADVATLRA
QLPADLRKAATSFDIITACMWRCRVSALQYGPDEVVRLIVAVNSRTKFDPPLTGYYGNGLMLPAAVTEAGKLVGSDLGYAVELV
REAKGINTEEYVRSAADFLVLNGRVHFVVSNTFLVSDLRRLIDLANMDWGWGKAVSGGPVDVGENVISFLATSKNSAGEEGA
VVPFCLPDSALGRFTSEVKKLVCFRPLENAAASNPDHGYMSRM
SEQ ID NO: 3 DNA sequence PipSynthase2 (1383 nucleotides; TAA-stop codon not
included)
ATGGCGCCTTCTTCTCAACTTGAGTTCAATGTGGTAAGGAAGCAACCGGAGCTTCTCCCTCCGGCCGAGCCAACTCCGTT
CGAGTGGAAAGAGTTGTCCGACATCGACGATCAAGACGGCTTACGCCTCCATATTTCTGGCTTTTTCATATACCCACCAT
CAACCATGTCCAATGTCGGCAGGGACAACGTCGCCCGGGACATCCGCCTGGGCTTGTCCAAGGCTATGGTCTTCTACTA
TCCATTAGCCGGCCGTATCCGCGAAGGCCCTAACAGAAAGCTCTCAGTAGAGTGCACCGGCGAGGGCATCATGTACTGC
GAGGCAGATGCCGACGTACGGCTAGAACAATTTGGCGATATCGGAGCACTCTCCATGCCATTCCCATTCATGGATAAGGT
GATGTTTGAAACAGTGGATGATGGTATCCTCGGAACCCCATTGATGTACTTCCAATCAACCCGCTTTATTTGCGGCGGGA
TCGTCATCGCCGGATGCTATAACCACGCCATAGCGGATGGGCTGGGGTTTTACATGTTTATGGCGGCCGCGGCCCAGCT
AGCGCGTGGCGCTGCCTCTCCGACGCCGCTCCCGGTGTGGCAGCGGGAGCGTCTCCTGTCGCGAGTGCCGCCTCGGG
TCACCTTCATTCACCATGAATACATTCACGACACCCCCAAAACTGAAACTGCAAATCCGCTGGACAACGAGCCATGGCCA
TTGCACTCAATCTTCTTCACCCGCGTTGACGTTGCCGCGCTGCGGGCTCAGCTGCCGGGCCATCTCCGAAAGTCCGCCA
CGTCCTTCGAGATCATCTCTGCCTGCCTATGGCGGTGCCGGACTGCGGCACTCCGTTTCGCACCCGACGAGTTGTCGCG
CTTGATCATCGCCGTCAACGCCCGAACCAAGTTCGGCCCACCTCTTCCACAAGGGTATTACGGCAACAGCATCATGCTCC
CGATGGTAGTGTCTGAAGCAGGAAAATTGGTGCTAAGCGGTCTCGGGTACGCGGTGGAGCTGGTGATTGAGGCCAAGG
GGAAGGTGACGGAGGAATACGTGAAGTCGGTGGCAGATTATTTGGTGCTCAATGGGAGGCCCCATTACGTAGTGACTAA
TACGTATCTGGTGTCTGACCTCCGACAACTGGTTGAAATGTCGAAATTTGATTGGGGGTGGGGGAAGCCGGCGTACGTT
GGGCCGGCGGATGTAGGGGAGAATGCCATAAGCTTCTTAGCAACTTTGAAAAACGGGGAAGAGGAGGGAGTGGTGGTG
CCCATTCGCTTACCCGAATCGGCCGTGGGAAGATTCAAGTCTGAGGTTAGCAAGATGGTTTCGTTTGGTTGCTTGGAGGA
CGTCAAGCCGAACCGAGATGGATACCTTTCCCGCATG
SEQ ID NO: 4 Amino acid sequence PipSynthase 2 (461 amino acids)
MAPSSQLEFNVVRKQPELLPPAEPTPFEWKELSDIDDQDGLRLHISGFFIYPPSTMSNVGRDNVARDIRLGLSKAMVFYYPLAG
RIREGPNRKLSVECTGEGIMYCEADADVRLEQFGDIGALSMPFPFMDKVMFETVDDGILGTPLMYFQSTRFICGGIVIAGCYNH
AIADGLGFYMFMAAAAQLARGAASPTPLPVWQRERLLSRVPPRVTFIHHEYIHDTPKTETANPLDNEPWPLHSIFFTRVDVAAL
RAQLPGHLRKSATSFEIISACLWRCRTAALRFAPDELSRLIIAVNARTKFGPPLPQGYYGNSIMLPMVVSEAGKLVLSGLGYAVE
LVIEAKGKVTEEYVKSVADYLVLNGRPHYVVTNTYLVSDLRQLVEMSKFDWGWGKPAYVGPADVGENAISFLATLKNGEEEGV
VVPIRLPESAVGRFKSEVSKMVSFGCLEDVKPNRDGYLSRM
SEQ ID NO: 5:
HXXXD-motif
SEQ ID NO: 6: amino acid sequence stretch
DWGWG
SEQ ID NO: 7: amino acid sequence stretch
DFGWG
SEQ ID NO: 8: Primer sequence
CATCATATGGCTTCTTCTCAGCTCGAATTC
SEQ ID NO: 9: Primer sequence
CATGGATCCTTACATGCGGGACATGTACCCATG
SEQ ID NO: 10: Primer sequence
CATCATATGGCGCCTTCTTCTCAACTTG
SEQ ID NO: 11: Primer sequence
CATGGATCCTTACATGCGGGAAAGGTATCCATC
SEQ ID NO: 12: Amino acid sequence
BAHD-like enzyme benzoyl-benzoate transferase from Clarkia breweri
SEQ ID NO: 13 Amino acid sequence
BAHD-like enzyme vinorine synthase from Rauwolfia serpentine
SEQ ID NO: 14: amino acid sequence stretch
LFLTAI
SEQ ID NO: 15: amino acid sequence stretch
GLML
SEQ ID NO: 16: amino acid sequence stretch
LHISGF
SEQ ID NO: 17: amino acid sequence stretch
SIML
SEQ ID NO: 18: amino acid sequence stretch
FLAT
SEQ ID NO: 19: Amino acid sequence of a benzyl alcohol O-benzoyltransferase-like
enzyme from Ziziphus jujuba
maqpptsltftvrrqqpelvapamptprelkplsdiddqeglrfqipviqfykydpsmegkdpvkvirralsqtivyyypfagrlregpqrklsvdctgegvm
fieadadvrleefgdalqppfpcleellfdvpgsggvldcpllliqvtrlkcggfifalrlnhtmsdaaglvqfmaaigemargacapsvtplwrrellnsrv
pprvtcthheydevadtkgtiiplddmthrsfffgptevsalrrfvppnlrkcstfevltaclwrcrtialqpdpeeevrvlcivnarakfnpplpegfygng
fafpvaltnagklcqkplgyalelvkkakddvteeymksladlmvikgrphftvvrsylvsdvtragfgevdfgwgkaayggpakggvgaipgvasfyipfkn
hkgesgivvpvclpapamerfvkeldgllntngvgagtsktfitsal
SEQ ID NO 20: Amino acid sequence of the capsaicin synthase PUN1 from Capsicum
annuum
mafalpsslvsvcnksfikpssltpstlrfhklsfidqslsnmyipcaffypkvqqrledsknsdelshiahllqtslsqtlvsyypyagklkdnatvdcndm
gaeflsvrikcsmseildhphaslaesivlpkdlpwannceggnllvvqvskfdcggiaisvcfshkigdgcsllnflndwssvtrdrttttlvpsprfvgds
vfstqkygslitpqilsdlnqcvqkrlifptdkldalrakvaeesgvknptraewsallfkcatkasssmlpsklvhflnirtmikprlprnaignlssifsi
eatnmqdmelptlvrnlrkevevaykkdqvegnelilevvesmregklpfenmdgyknvytcsnlckypyytvdfgwgrpervclgngpsknafflkdykagq
gvearvmlhkqqmseferneellefia
SEQ ID NO: 21: Amino acid sequence of the shikimate O-hydroxycinnamoyltransferase
from Arabidopsis thaliana
MKINIRDSTMVRPATETPITNLWNSNVDLVIPRFHTPSVYFYRPTGASNFFDPQVMKEALSKALVPFYPMAGRLK
RDDDGRIEIDCNGAGVLFVVADTPSVIDDFGDFAPTLNLRQLIPEVDHSAGIHSFPLLVLQVTFFKCGGASLGVG
MQHHAADGFSGLHFINTWSDMARGLDLTIPPFIDRTLLRARDPPQPAFHHVEYQPAPSMKIPLDPSKSGPENTT
VSIFKLTRDQLVALKAKSKEDGNTVSYSSYEMLAGHVWRSVGKARGLPNDQETKLYIATDGRSRLRPQLPPGYF
GNVIFTATPLAVAGDLLSKPTWYAAGQIHDFLVRMDDNYLRSALDYLEMQPDLSALVRGAHTYKCPNLGITSWV
RLPIYDADFGWGRPIFMGPGGIPYEGLSFVLPSPTNDGSLSVAIALQSEHMKLFEKFLFEI

1. A polynucleotide the nucleotide sequence of which is:
(a) the nucleotide sequence defined in SEQ ID NO: 1, or
(b) a nucleotide sequence of at least 93% sequence identity to SEQ ID NO: 1, or
(c) a nucleotide sequence encoding an amino acid sequence comprising or consisting of the amino acid sequence defined in SEQ ID NO: 2, or
(d) a nucleotide sequence encoding an amino acid sequence of at least 70% sequence identity to the amino acid sequence defined in SEQ ID NO: 2, or
(e) a nucleotide sequence encoding an amino acid sequence of at least 75% sequence similarity to the amino acid sequence defined in SEQ ID NO: 2, or
(f) a nucleotide sequence encoding an amino acid sequence of from 1 to 138 amino acid substitutions, additions, insertions and/or deletions compared to the amino acid sequence defined in SEQ ID NO: 2, or
(g) a fragment of the nucleotide sequence defined in any one of items (a) to (f), the fragment being at most 42 nucleotides shorter due to 5′- and/or 3′-terminal deletions of the nucleotide sequence defined in any one of items (a) to (f), or
(a′) the nucleotide sequence defined in SEQ ID NO: 3, or
(b′) a nucleotide sequence of at least 93% sequence identity to SEQ ID NO: 3, or
(c′) a nucleotide sequence encoding an amino acid sequence comprising or consisting of the amino acid sequence defined in SEQ ID NO: 4, or
(d′) a nucleotide sequence encoding an amino acid sequence of at least 70% sequence identity to the amino acid sequence defined in SEQ ID NO: 4, or
(e′) a nucleotide sequence encoding an amino acid sequence of at least 75% sequence similarity to the amino acid sequence defined in SEQ ID NO: 4, or
(f′) a nucleotide sequence encoding an amino acid sequence of from 1 to 138 amino acid substitutions, additions, insertions and/or deletions compared to the amino acid sequence defined in SEQ ID NO: 4, or
(g′) a fragment of the nucleotide sequence defined in any one of items (a′) to (f′), the fragment being at most 42 nucleotides shorter due to 5′- and/or 3′-terminal deletions of the nucleotide sequence defined in any one of items (a′) to (f′).

2. (canceled)

3. Nucleic acid molecule, recombinant construct, or vector comprising the polynucleotide defined in claim 1, optionally further comprising a polynucleotide, nucleic acid or gene encoding a CoA-ligase.

4. A prokaryotic or eukaryotic cell comprising the nucleic acid molecule or vector according to claim 3, and optionally a further polynucleotide encoding a CoA-ligase.

5. A protein, or protein composition comprising said protein, said protein or protein composition having a catalytic activity for converting piperoyl-CoA and piperidine to piperine with a catalytic activity of at least 0.8, preferably at least 2.4, and most preferably at least 5.0 nkat/mg of said protein or protein composition, respectively, at 30° C. in 30 mM Tris-HCl buffer pH 8.0, containing 1 mM dithiothreitol (DTT), with substrate concentrations of 500 μM piperoyl-CoA and 20 mM piperidine.

6. A protein comprising a polypeptide the amino acid sequence of which
(i) is or comprises the amino acid sequence defined in SEQ ID NO: 2, or
(ii) comprises or consists of an amino acid sequence that has at least 70% sequence identity to the amino acid sequence defined in SEQ ID NO: 2, or
(iii) comprises or consists of an amino acid sequence that has at least 75% sequence similarity to the amino acid sequence defined in SEQ ID NO: 2, or
(iv) comprises or consists of an amino acid sequence of from 1 to 138 amino acid substitutions, additions, insertions and/or deletions compared to the amino acid sequence defined in SEQ ID NO: 2, or
(v) is a fragment of the amino acid sequence defined in any one of items (i) to (iv), the fragment being at most 14 amino acid residues shorter due to N- and/or C-terminal deletions of the amino acid sequence defined in any one of items (i) to (iv), or (i′) is or comprises the amino acid sequence defined in SEQ ID NO: 4, or
(ii′) comprises or consists of an amino acid sequence that has at least 70% sequence identity to the amino acid sequence defined in SEQ ID NO: 4, or
(iii′) comprises or consists of an amino acid sequence that has at least 75% sequence similarity to the amino acid sequence defined in SEQ ID NO: 4, or
(iv′) comprises or consists of an amino acid sequence of from 1 to 138 amino acid substitutions, additions, insertions and/or deletions compared to the amino acid sequence defined in SEQ ID NO: 4.

7. (canceled)

8. The protein according to claim 5, comprising a polypeptide comprising the following amino acid residues or amino acid sequence stretches in the one-letter code:
L at the position corresponding to amino acid residue 42 in SED ID NO: 2 or at the position corresponding to amino acid residue 43 in SED ID NO: 4,
DVG at the position corresponding to amino acid residues 396 to 398 in SED ID NO: 2 or at the position corresponding to amino acid residues 400 to 402 in SED ID NO: 4,
FLAT at the position corresponding to amino acid residues 404 to 407 in SED ID NO: 2 or at the position corresponding to amino acid residues 408 to 411 in SED ID NO: 4,
ML at the position corresponding to amino acid residues 312 to 313 in SED ID NO: 2 or at the position corresponding to amino acid residues 316 to 317 in SED ID NO: 4,
N at the position corresponding to amino acid residue 366 in SED ID NO: 2 or at the position corresponding to amino acid residue 370 in SED ID NO: 4,
HXXXD at the position corresponding to amino acid residues 168 to 172 in SED ID NO: 2 or at the position corresponding to amino acid residues 168 to 172 in SED ID NO: 4,
DWGWG at the position corresponding to amino acid residues 383 to 387 in SED ID NO: 2 or at the position corresponding to amino acid residues 387 to 391 in SED ID NO: 4,
F at the position corresponding to amino acid residue 130 in SEQ ID NO: 2 or at the position corresponding to amino acid residue 129 in SEQ ID NO: 4, and/or
K at the position corresponding to amino acid residue 133 in SEQ ID NO: 2 or at the position corresponding to amino acid residue 132 in SEQ ID NO: 4.

9. The protein according to claim 5, wherein the protein comprises a polypeptide comprising one or more or all of the following amino acid residues or amino acid sequence stretches in the one-letter code:
LFLTAI at the position corresponding to amino acid residues 42 to 47 in SEQ ID NO: 2,
GLML at the position corresponding to amino acid residues 310 to 313 in SEQ ID NO: 2,
SN at the position corresponding to amino acid residues 365 to 366 in SEQ ID NO: 2, and/or
LID at the position corresponding to amino acid residues 376 to 378 in SEQ ID NO: 2.

10. The protein according to claim 5, wherein the protein comprises a polypeptide comprising one or more or all of the following amino acid residues or amino acid sequence stretches in the one-letter code:
LHISGF at the position corresponding to amino acid residues 43 to 48 in SEQ ID NO: 4,
SIML at the position corresponding to amino acid residues 314 to 317 in SEQ ID NO: 4,
TN at the position corresponding to amino acid residues 369 to 370 in SEQ ID NO: 4, and/or
LVE at the position corresponding to amino acid residues 380 to 382 in SEQ ID NO: 4.

11. The protein according to claim 5, wherein said protein is an enzyme having a catalytic activity of converting piperoyl-CoA and piperidine to isopiperine and/or chavicine with a catalytic activity of at least 0.56, preferably at least 1.6, more preferably at least 2.4, even more preferably at least 3.2, and most preferably of at least 9.6 nkat/mg total protein at pH 8.0 at 30° C. in 30 mM Tris-HCl buffer containing 1 mM dithiothreitol (DTT); and/or
wherein said protein is an enzyme having a catalytic activity of converting piperoyl-CoA and piperidine to piperine with a catalytic activity of at least 0.14, preferably at least 0.4, more preferably at least 0.8, even more preferably at least 2.4 nkat/mg total protein at pH 8.0 at 30° C. in 30 mM Tris-HCl buffer containing 1 mM dithiothreitol (DTT).

12. The protein according to claim 6, wherein said protein is an enzyme having a catalytic activity for converting piperoyl-CoA and piperidine to piperine with a catalytic activity of at least 0.14, preferably at least 0.4, more preferably at least 0.8, even more preferably at least 2.4, and most preferably at least 5.0 nkat/mg total protein at pH 8.0 at 30° C. in 30 mM Tris-HCl buffer containing 1 mM dithiothreitol (DTT).

13. A process of producing an amide from an acyl-CoA and an amine that is a primary or secondary amine, comprising incubating the acyl-CoA and the amine in the presence of a protein according to claim 5, optionally followed by isolating the amide produced.

14. The process according to claim 13, wherein the acyl-CoA is a C₁-C₂₀ acyl-CoA, preferably a C₃-C₁₈ acyl-CoA, more preferably a C₄-C₁₆ acyl-CoA, and most preferably a C₅-C₁₄ acyl-CoA, preferably the acyl-CoA is a C₁-C₂₀ alkanoyl-CoA or C₁ to C₁₄ alkanoyl-CoA; and/or
wherein the amine is a C₁-C₁₄ amine, preferably a C₂-C₁₂ amine, preferably a C₃-C₁₀ amine, and most preferably a C₅-C₈ amine; and/or the amine is a hydrocarbylamine, a heterohydrocarbylamine, a di-N-hydrocarbylamine, a N-hydrocarbyl-N-heterohydrocarbyl-amine, or a di-N-heterohydrocarbylamine, said heterohydrocarbyl moiety having from 1 to 3 heteroatoms selected from O, N or S, preferably O; or has 1 or 2 heteroatoms selected from O, N or S, preferably O.

15. The process according to claim 13, wherein the amine is a substituted or unsubstituted: alkylamine, di-N-alkylamine, cycloalkyl-amine, heterocycloalkyl-amine, N-alkyl-N-cycloalkyl-amine, cycloalkenylamine, N-alkyl-N-cycloalkenyl-amine, N-cycloalkylalkylamine, N-alkyl-N-cycloalkylalkylamine, aniline, N-alkyl-aniline, N-phenylalkylamine, N-alkyl-N-phenylalkyl-amine, wherein these substituted or unsubstituted compounds preferably have from 2 to 14 carbon atoms; and/or
wherein the amine is a substituted or unsubstituted four-, five-, six-, or seven-membered nitrogen-containing ring such as azetidine, pyrrole, pyrroline, pyrrolidine, imidazole, imidazoline, oxazole, oxazoline, oxazolidine, pyridine, dihydropyridine, piperidine, piperazine, or morpholine, wherein these substituted or unsubstituted compounds preferably have from 2 to 14 carbon atoms.

16. The process according to claim 13, wherein the acyl-CoA is a compound of the following general formula (1): 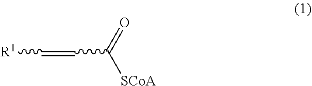
wherein R¹ is a C₁-C₁₇ hydrocarbyl group or C₂-C₁₆ heterohydrocarbyl group, and SCoA is a CoA group; or
wherein the acyl-CoA is a compound of the following general formula (5): 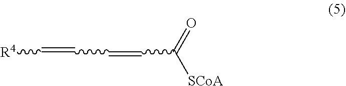
wherein R⁴ is a C₁-C₁₅ hydrocarbyl group or C₂-C₁₄ heterohydrocarbyl group, and SCoA is a CoA group; or
wherein the acyl-CoA is a compound of the following general formula (9): 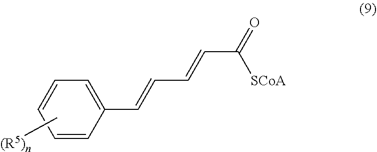
wherein n is an integer of from 1 to 4, preferably 1 or 2, multiple groups R⁵ may be the same or different and R⁵ is selected from C₁ to C₆ alkyl, C₂ to C₆ alkenyl, C₂ to C₆ alkynyl, C₁ to C₆ alkoxy, hydroxy, or hydroxy-C₁ to C₆-alkyl, and sulfhydryl, or two R⁵ at adjacent carbon atoms of the phenyl ring together form a five- or six-membered ring, such as a 3,4-methylene dioxy group, and SCoA is a CoA group.

17. The process according to claim 13, wherein the acyl-CoA is piperoyl-CoA, 5-phenylpentanoyl-CoA, or 3,4-methylenedioxy cinnamoyl-CoA, and the primary or secondary amine is selected from the group consisting of piperidine, pyrrolidine, isobutylamine, benzylamine, 4-aminomethylpiperidine, and 1,7-diamino-n-heptane; or piperine or chavicine is produced from piperoyl-CoA and piperidine.

18. The process according to claim 13, wherein the acyl-CoA is piperoyl-CoA and the amine is piperidine.

19. A process of producing an amide from a carboxylic acid and an amine that is a primary or secondary amine, comprising incubating the carboxylic acid and the amine in the presence of a CoA ligase, coenzyme A, and a protein according to claim 5, optionally followed by isolating the amide produced.


SEQUENCE LISTING (21 sequences)

SEQ ID NO: 1 - DNA, 1380 bp, Artificial Sequence
  CDS
atggcttcttctcagctcgaattcaatgtggagaggaagcaaccggagcttctcggcccggcagaaccaactccctacgagttgaaggagctgtcggacatcgacgaccaagacggtgtccgcctctttttgactgccattttcatataccccccaccaaccaagacatccatgcccacccgcaaaaccgacccagctagcgacatccgccggggcctatccaaagctatggtctactactacccttttgccggccgtataagagaaggccccaacaggaagctatccgttgactgcaccggcgagggaatcgtgttctgcgaggccgacgccgacattcggctagatggattgggtgacgtcgaggtgctccgtccgccctacccattcatagacaagatgacgctgggagaggggagtgccatcctcggcgcacccttggtgtacgtgcaggtgacccgctttgcctgcggtgggttcatcatcaccggacgtttcaaccatgtcatggccgatgcgccggggtttaccatgtttatgaaggccgcggccgacctcgcacgcggagccaccgtcccgatgccgctaccggtgtgggagcgggagcgttaccggtcgcgagtgccgccccgggtcaccttcgcccaccatgaatacatgcacgtcgacgatcccccgcccagacctacgagcgagccatggtcattgcactccgccttcttcaccaaggctgacgtcgccacgctccgggcacagctgccggccgaccttcggaaagcggcaacctcctttgatatcatcaccgcctgcatgtggcggtgccgggtgtcggcactccagtacgggccggacgaggtggttcgactgatcgtcgccgtcaactcccggaccaagttcgaccctcctctaacagggtactacggtaacggcctcatgctcccggccgccgtgactgaagccgggaagctagtcgggagcgaccttgggtacgcggtggagctggtgagggaggccaaggggaaggtgacggaggagtacgtgcggtcggcggcggacttcttggtgctcaatgggagggtccatttcgtggtgagcaatacgttcttggtgtcggatcttcggcggttgattgatttggcgaatatggattggggatgggggaaggcggtgtccggtgggccggtggatgtaggggagaatgtgataagcttcttggcgacgtcgaagaacagtgcaggggaggaaggagcggtggttcctttctgcttgccggattctgccctgggaaggttcacgtcggaggttaagaagctggtttgttttcgtccgttggagaacgcggcggcgtcgaatccagatcatgggtacatgtcccgcatg

SEQ ID NO: 2 - protein, 460 aa, Piper nigrum
MASSQLEFNVERKQPELLGPAEPTPYELKELSDIDDQDGVRLFLTAIFIYPPPTKTSMPTRKTDPASDIRRGLSKAMVYYYPFAGRIREGPNRKLSVDCTGEGIVFCEADADIRLDGLGDVEVLRPPYPFIDKMTLGEGSAILGAPLVYVQVTRFACGGFIITGRFNHVMADAPGFTMFMKAAADLARGATVPMPLPVWERERYRSRVPPRVTFAHHEYMHVDDPPPRPTSEPWSLHSAFFTKADVATLRAQLPADLRKAATSFDIITACMWRCRVSALQYGPDEVVRLIVAVNSRTKFDPPLTGYYGNGLMLPAAVTEAGKLVGSDLGYAVELVREAKGKVTEEYVRSAADFLVLNGRVHFVVSNTFLVSDLRRLIDLANMDWGWGKAVSGGPVDVGENVISFLATSKNSAGEEGAVVPFCLPDSALGRFTSEVKKLVCFRPLENAAASNPDHGYMSRM

SEQ ID NO: 3 - DNA, 1383 bp, Artificial Sequence
  CDS
atggcgccttcttctcaacttgagttcaatgtggtaaggaagcaaccggagcttctccctccggccgagccaactccgttcgagtggaaagagttgtccgacatcgacgatcaagacggcttacgcctccatatttctggctttttcatatacccaccatcaaccatgtccaatgtcggcagggacaacgtcgcccgggacatccgcctgggcttgtccaaggctatggtcttctactatccattagccggccgtatccgcgaaggccctaacagaaagctctcagtagagtgcaccggcgagggcatcatgtactgcgaggcagatgccgacgtacggctagaacaatttggcgatatcggagcactctccatgccattcccattcatggataaggtgatgtttgaaacagtggatgatggtatcctcggaaccccattgatgtacttccaatcaacccgctttatttgcggcgggatcgtcatcgccggatgctataaccacgccatagcggatgggctggggttttacatgtttatggcggccgcggcccagctagcgcgtggcgctgcctctccgacgccgctcccggtgtggcagcgggagcgtctcctgtcgcgagtgccgcctcgggtcaccttcattcaccatgaatacattcacgacacccccaaaactgaaactgcaaatccgctggacaacgagccatggccattgcactcaatcttcttcacccgcgttgacgttgccgcgctgcgggctcagctgccgggccatctccgaaagtccgccacgtccttcgagatcatctctgcctgcctatggcggtgccggactgcggcactccgtttcgcacccgacgagttgtcgcgcttgatcatcgccgtcaacgcccgaaccaagttcggcccacctcttccacaagggtattacggcaacagcatcatgctcccgatggtagtgtctgaagcaggaaaattggtgctaagcggtctcgggtacgcggtggagctggtgattgaggccaaggggaaggtgacggaggaatacgtgaagtcggtggcagattatttggtgctcaatgggaggccccattacgtagtgactaatacgtatctggtgtctgacctccgacaactggttgaaatgtcgaaatttgattgggggtgggggaagccggcgtacgttgggccggcggatgtaggggagaatgccataagcttcttagcaactttgaaaaacggggaagaggagggagtggtggtgcccattcgcttacccgaatcggccgtgggaagattcaagtctgaggttagcaagatggtttcgtttggttgcttggaggacgtcaagccgaaccgagatggatacctttcccgcatg

SEQ ID NO: 4 - protein, 461 aa, Piper nigrum
MAPSSQLEFNVVRKQPELLPPAEPTPFEWKELSDIDDQDGLRLHISGFFIYPPSTMSNVGRDNVARDIRLGLSKAMVFYYPLAGRIREGPNRKLSVECTGEGIMYCEADADVRLEQFGDIGALSMPFPFMDKVMFETVDDGILGTPLMYFQSTRFICGGIVIAGCYNHAIADGLGFYMFMAAAAQLARGAASPTPLPVWQRERLLSRVPPRVTFIHHEYIHDTPKTETANPLDNEPWPLHSIFFTRVDVAALRAQLPGHLRKSATSFEIISACLWRCRTAALRFAPDELSRLIIAVNARTKFGPPLPQGYYGNSIMLPMVVSEAGKLVLSGLGYAVELVIEAKGKVTEEYVKSVADYLVLNGRPHYVVTNTYLVSDLRQLVEMSKFDWGWGKPAYVGPADVGENAISFLATLKNGEEEGVVVPIRLPESAVGRFKSEVSKMVSFGCLEDVKPNRDGYLSRM

SEQ ID NO: 5 - protein, 5 aa, Artificial Sequence
  sequence motif
  misc_feature (2)..(4): Xaa can be any naturally occurring amino acid
HXXXD

SEQ ID NO: 6 - protein, 5 aa, Artificial Sequence
  sequence motif
DWGWG

SEQ ID NO: 7 - protein, 5 aa, Artificial Sequence
  sequence motif
DFGWG

SEQ ID NO: 8 - DNA, 30 bp, Artificial Sequence
  Oligo
catcatatggcttcttctcagctcgaattc

SEQ ID NO: 9 - DNA, 33 bp, Artificial Sequence
  Oligo
catggatccttacatgcgggacatgtacccatg

SEQ ID NO: 10 - DNA, 28 bp, Artificial Sequence
  Oligo
catcatatggcgccttcttctcaacttg

SEQ ID NO: 11 - DNA, 33 bp, Artificial Sequence
  Oligo
catggatccttacatgcgggaaaggtatccatc

SEQ ID NO: 12 - protein, 456 aa, Clarkia breweri
MAHDQSLSFEVCRRKPELIRPAKQTPHEFKKLSDVEDQEGLRFQIPVIQFYKHNNESMQERDPVQVIREGIARALVYYYPFAGRLREVDGRKLVVECTGEGVMFIEADADVTLEQFGDALQPPFPCFDQLLFDVPGSGGILDSPLLLIQVTRLKCGSFIFALRLNHTMADAAGIVLFMKAVGEMARGAATPSTLPVWDRHILNARVPPQVTFNHREYEEVKGTIFTPFDDLAHRSFFFGSTEISAMRKQIPPHLRSCSTTIEVLTACLWRCRTLAIKPNPDEEVRMICIVNARSKFNPPLPDGYYGNAFAIPAAVTTAGKLCNNPLGFALELIRKAKREVTEEYMHSVADLMVATGRPHFTVVNTYLVSDVTRAGFGEVDFGWGEAVYGGPAKGGVGVIPGVTSFYIPLRNRQGEKGIVLPICLPSAAMEIFAEALNNTLNGKEIEIAKHFTQSSL

SEQ ID NO: 13 - protein, 421 aa, Rauwolfia serpentina
MAPQMEKVSEELILPSSPTPQSLKCYKISHLDQLLLTCHIPFILFYPNPLDSNLDPAQTSQHLKQSLSKVLTHFYPLAGRINVNSSVDCNDSGVPFVEARVQAQLSQAIQNVVELEKLDQYLPSAAYPGGKIEVNEDVPLAVKISFFECGGTAIGVNLSHKIADVLSLATFLNAWTATCRGETEIVLPNFDLAARHFPPVDNTPSPELVPDENVVMKRFVFDKEKIGALRAQASSASEEKNFSRVQLVVAYIWKHVIDVTRAKYGAKNKFVVVQAVNLRSRMNPPLPHYAMGNIATLLFAAVDAEWDKDFPDLIGPLRTSLEKTEDDHNHELLKGMTCLYELEPQELLSFTSWCRLGFYDLDFGWGKPLSACTTTFPKRNAALLMDTRSGDGVEAWLPMAEDEMAMLPVELLSLVDSDFSK

SEQ ID NO: 14 - protein, 6 aa, Artificial Sequence
  sequence motif
LFLTAI

SEQ ID NO: 15 - protein, 4 aa, Artificial Sequence
  sequence motif
GLML

SEQ ID NO: 16 - protein, 6 aa, Artificial Sequence
  sequence motif
LHISGF

SEQ ID NO: 17 - protein, 4 aa, Artificial Sequence
  sequence motif
SIML

SEQ ID NO: 18 - protein, 4 aa, Artificial Sequence
  sequence motif
FLAT

SEQ ID NO: 19 - protein, 459 aa, Ziziphus jujuba
MAQPPTSLTFTVRRQQPELVAPAMPTPRELKPLSDIDDQEGLRFQIPVIQFYKYDPSMEGKDPVKVIRRALSQTLVYYYPFAGRLREGPQRKLSVDCTGEGVMFIEADADVRLEEFGDALQPPFPCLEELLFDVPGSGGVLDCPLLLIQVTRLKCGGFIFALRLNHTMSDAAGLVQFMAAIGEMARGACAPSVTPLWRRELLNSRVPPRVTCTHHEYDEVADTKGTIIPLDDMTHRSFFFGPTEVSALRRFVPPNLRKCSTFEVLTACLWRCRTIALQPDPEEEVRVLCIVNARAKFNPPLPEGFYGNGFAFPVALTNAGKLCQKPLGYALELVKKAKDDVTEEYMKSLADLMVIKGRPHFTVVRSYLVSDVTRAGFGEVDFGWGKAAYGGPAKGGVGAIPGVASFYIPFKNHKGESGIVVPVCLPAPAMERFVKELDGLLNTNGVGAGTSKTFITSAL

SEQ ID NO: 20 - protein, 440 aa, Capsicum annuum
MAFALPSSLVSVCNKSFIKPSSLTPSTLRFHKLSFIDQSLSNMYIPCAFFYPKVQQRLEDSKNSDELSHIAHLLQTSLSQTLVSYYPYAGKLKDNATVDCNDMGAEFLSVRIKCSMSEILDHPHASLAESIVLPKDLPWANNCEGGNLLVVQVSKFDCGGIAISVCFSHKIGDGCSLLNFLNDWSSVTRDRTTTTLVPSPRFVGDSVFSTQKYGSLITPQILSDLNQCVQKRLIFPTDKLDALRAKVAEESGVKNPTRAEVVSALLFKCATKASSSMLPSKLVHFLNIRTMIKPRLPRNAIGNLSSIFSIEATNMQDMELPTLVRNLRKEVEVAYKKDQVEQNELILEVVESMREGKLPFENMDGYKNVYTCSNLCKYPYYTVDFGWGRPERVCLGNGPSKNAFFLKDYKAGQGVEARVMLHKQQMSEFERNEELLEFIA

SEQ ID NO: 21 - protein, 433 aa, Arabidopsis thaliana
MKINIRDSTMVRPATETPITNLWNSNVDLVIPRFHTPSVYFYRPTGASNFFDPQVMKEALSKALVPFYPMAGRLKRDDDGRIEIDCNGAGVLFVVADTPSVIDDFGDFAPTLNLRQLIPEVDHSAGIHSFPLLVLQVTFFKCGGASLGVGMQHHAADGFSGLHFINTWSDMARGLDLTIPPFIDRTLLRARDPPQPAFHHVEYQPAPSMKIPLDPSKSGPENTTVSIFKLTRDQLVALKAKSKEDGNTVSYSSYEMLAGHVWRSVGKARGLPNDQETKLYIATDGRSRLRPQLPPGYFGNVIFTATPLAVAGDLLSKPTWYAAGQIHDFLVRMDDNYLRSALDYLEMQPDLSALVRGAHTYKCPNLGITSWVRLPIYDADFGWGRPIFMGPGGIPYEGLSFVLPSPTNDGSLSVAIALQSEHMKLFEKFLFEI